(19) United States
(12) Patent Application Publication
(10) Pub. No.: US 2025/0046429 A1
(43) Pub. Date: Feb. 6, 2025

(54) SELF-SUPERVISED LEARNING FOR MEDICAL IMAGE ANALYSIS

(71) Applicants: Fatemeh Haghighi, Tempe, AZ (US); Mohammad Reza Hosseinzadeh Taher, Tempe, AZ (US); Jianming Liang, Scottsdale, AZ (US)

(72) Inventors: Fatemeh Haghighi, Tempe, AZ (US); Mohammad Reza Hosseinzadeh Taher, Tempe, AZ (US); Jianming Liang, Scottsdale, AZ (US)

(73) Assignee: Arizona Board of Regents on Behalf of Arizona State University, Tempe, AZ (US)

(21) Appl. No.: 18/792,461

(22) Filed: Aug. 1, 2024

Related U.S. Application Data
(60) Provisional application No. 63/530,224, filed on Aug. 1, 2023.

(51) Int. Cl. G16H 30/40

(52) U.S. Cl. CPC G16H 30/40

(57) ABSTRACT

Examples of implementation and training of artificial intelligence models developed with self-supervised learning for medical image analysis are disclosed. The models as trained leverage training from discriminative learning, restorative learning, and adversarial learning in a unified manner to glean complementary visual information from unlabeled medical images for fine-grained semantic representation learning. The models as trained provide features suitable for generalizable representation of medical input images many organs, diseases, and modalities.

### CROSS REFERENCE TO RELATED APPLICATIONS

This is a non-provisional application that claims benefit to U.S. Provisional Application Ser. No. 63/530,224, filed on Aug. 1, 2023, which is herein incorporated by reference in its entirety.

### GOVERNMENT SUPPORT

This invention was made with government support under R01 HL128785 awarded by the National Institutes of Health and under 1548562 awarded by the National Science Foundation. The government has certain rights in the invention.

### FIELD

The present disclosure generally relates to artificial intelligence applications for medical image analysis; and in particular to self-supervised learning for medical image analysis using a training framework that unifies discriminative, restorative, and adversarial learning for fine-grained semantic representation learning and implementation.

### BACKGROUND

Achieving outstanding performance on medical vision tasks requires a higher level of feature granularity compared to the computer vision tasks. Photographic images, particularly those found in ImageNet, typically consist of large foreground objects with distinct discriminative parts that are set against varying backgrounds. Thus, image recognition tasks in photographic images are primarily based on high-level features captured from discriminative regions. As a result, in computer vision, discriminative learning is more preferred. Conversely, medical images acquired through specific imaging protocols exhibit consistent anatomical structures with clinically relevant information dispersed over the entire image. In particular, high-level structural information, i.e., anatomical structures and their relative spatial orientations are essential for the identification of normal anatomy and various disorders. Furthermore, medical tasks demand heightened attention to fine-grained details within images, as the identification of diseases, organ delineation, and lesion isolation depend on subtle, local variations in texture.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1 is a chart illustrating components of the self-supervised learning framework described herein, referenced herein as “DiRA”.

FIG. 2 is a series of photographic images illustrating the difference in possible features that can be utilized/extracted from general photographic images (a) medical images (b).

FIG. 3 is an illustration of the self-supervised learning framework (DiRA) described herein that aims to learn generalizable image representations by uniting three learning components: discriminative, restorative, and adversary.

FIG. 4 is a series of graphs ((a)-(f)) illustrating comparison with discriminative learning methods.

FIG. 5 is a series of graphs ((a)-(f)) illustrating weakly-supervised pathology localization accuracy under IoU thresholds.

FIG. 6 is three sets of images ((a)-(c)) illustrating visualization of Grad-CAM heatmaps for (a) MoCo-v2 vs. DiRA_{MoCo-v2}, (b) Barlow Twins vs. DiRA_{Barlow Twins}, and (c) SimSiam vs. DiRA_{SimSiam}.

FIG. 7 is a series of reports ((a)-(f)) showing comparison with restorative self-supervised learning: we apply DiRA to MAE, a representative of ViT-based masked image modeling self-supervised methods.

FIG. 8 is a series of graphs ((a)-(c)) illustrating that DiRA_{TransVW} exhibits greater label efficiency in 3D downstream tasks, reducing annotation costs by 60%, 57%, and 57% in comparison with training from scratch, and 87%, 16%, and 26% compared with TransVW in lung nodule segmentation, PE FPR, and lung nodule FPR, respectively.

FIG. 9 is series of graphs illustrating comparison of feature distance distributions: incorporating fine-grained representation learning into instance discrimination SSL methods leads to enlarged feature distances compared with the original methods.

FIG. 10 shows three sets related to an ablation study on loss coefficient: various scales for the (a) discriminative loss coefficient (λ_{dis}), (b) restorative loss coefficient (λᵣₑₛ), and (c) adversarial loss coefficients (λ_{adv}) were examined using the DiRA_{MoCo-v2} model.

FIG. 11 illustrates an ablation study on the impact of pretraining data scale on the transfer performance of contrastive learning—MoCo-v2 models pretrained with varying amounts of unlabeled data, ranging from 86K to 4.8M, across five downstream tasks were evaluated and it was shown that increasing the volume of pretraining data may not necessarily yield additional benefits to downstream performance.

FIG. 12 is a visualization of Grad-CAM heatmaps for MoCo-v2 vs. DiRA_{MoCo-v2}.

FIG. 13 is a visualization of Grad-CAM heatmaps for Barlow Twins vs. DiRA_{Barlow Twins}.

FIG. 14 is a visualization of Grad-CAM heatmaps for SimSiam vs. DiRA_{SimSiam}.

FIG. 15 is an exemplary simplified block diagram of a computing device that may be configured to implement various methodologies described herein.

Corresponding reference characters indicate corresponding elements among the view of the drawings. The headings used in the figures do not limit the scope of the claims.

### DETAILED DESCRIPTION

Aspects of the present inventive concept relate to example implementations for self-supervised learning for medical image analysis. Discriminative, restorative, and adversarial learning have proven beneficial for self-supervised learning schemes in computer vision and medical imaging. Existing efforts, however, fail to capitalize on the potentially synergistic effects these methods may offer in a ternary setup, which, it is envisioned can significantly benefit deep semantic representation learning. Towards this end, DiRA was developed and is described herein, believed to be the first framework that unites discriminative, restorative, and adversarial learning in a unified manner to collaboratively glean complementary visual information from unlabeled medical images for fine-grained semantic representation learning. Extensive experiments demonstrate that DiRA: (1) encourages collaborative learning among three learning ingredients, resulting in more generalizable representation across organs, diseases, and modalities; (2) outperforms fully supervised ImageNet models and increases robustness in small data regimes, reducing annotation cost across multiple medical imaging applications; (3) learns fine-grained semantic representation, facilitating accurate lesion localization with only image-level annotation; (4) improves reusability of low/mid-level features; and (5) enhances restorative self-supervised approaches, revealing that DiRA is a general framework for united representation learning.

### 1. INTRODUCTION

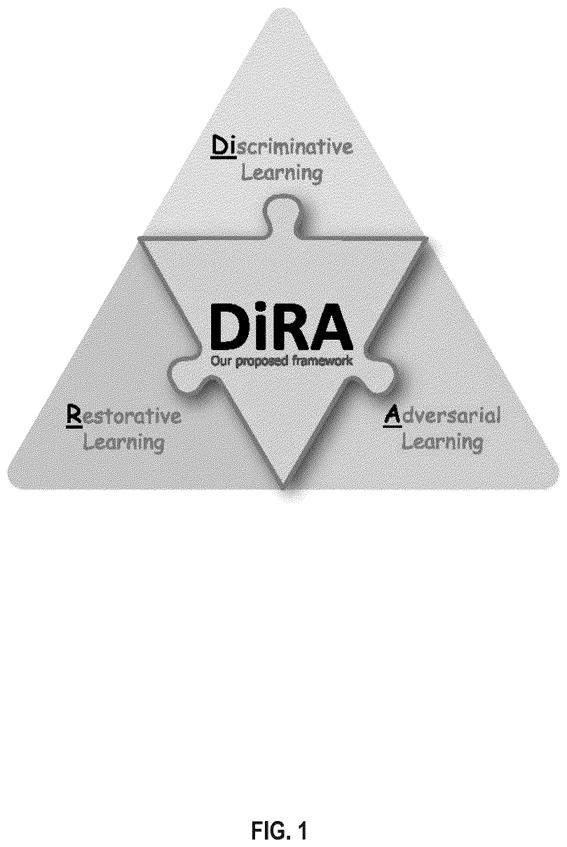

Self-supervised learning (SSL) aims to learn generalizable representations without relying on expert annotations. The mainstream self-supervised representation learning approaches can be categorized into three groups: (1) discriminative learning, which utilizes encoders to distinguish instances from different (pseudo) classes; (2) restorative learning, which employs encoder-decoder models to reconstruct original images from their distorted versions; and (3) adversarial learning, which leverages adversary models to enhance restorative learning. Despite the significant contributions of discriminative, restorative, and adversarial learning to the performance of SSL individually (Chen et al., 2020b; He et al., 2022; Zbontar et al., 2021; Grill et al., 2020) or in pairs (Haghighi et al., 2021; Tao et al., 2020; Zhou et al., 2021a; Hosseinzadeh Taher et al., 2022), the simultaneous exploitation of all three learning ingredients in a SSL schema remains unexploited (see FIG. 1). Therefore, it is desired to determine if discriminative, restorative, and adversarial learning can be seamlessly integrated into a single framework to foster collaborative learning for deep semantic representation, yielding more powerful SSL models capable of excelling across a broad range of applications?

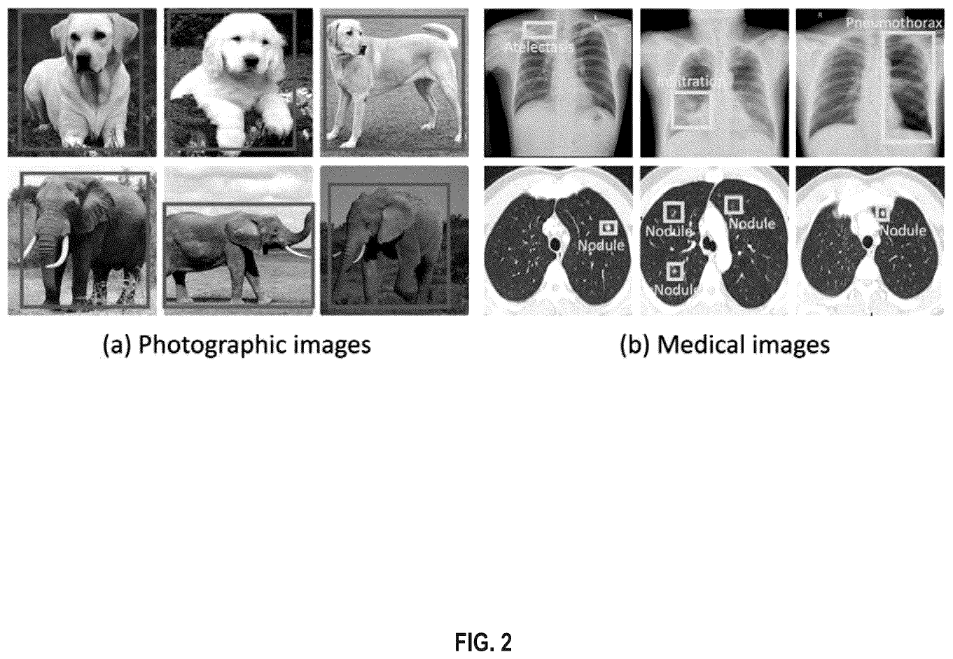

Achieving outstanding performance for medical vision tasks requires a higher level of feature granularity compared with computer vision tasks. This requirement stems from the marked disparities between photographic and medical images, leading to significant impacts on the effectiveness of discriminative, restorative, and adversarial learning techniques for photographic and for medical images. Photographic images, particularly those found in ImageNet, typically consist of large foreground objects with distinct discriminative parts that are set against varying backgrounds. For example, FIG. 2 illustrates that (a) photographic images commonly contain large foreground objects with distinct discriminative parts (e.g., tusks of an elephant); and in contrast, (b) medical images acquired from a particular imaging protocol often exhibit remarkable similarity in anatomy (e.g. lungs), with semantic information dispersed over the entire image. Consequently, recognition tasks in photographic images primarily require high-level features, whereas medical tasks demand holistic fine-grained discriminative features captured throughout images. Thus, image recognition tasks in photographic images are primarily based on high-level features captured from discriminative regions. Therefore, for computer vision tasks, discriminative learning is preferred, as evidenced by the state-of-the-art performance of discriminative SSL methods, notably instance discrimination learning (Chen et al., 2020b; Chen and He, 2021; Zbontar et al., 2021; Grill et al., 2020; He et al., 2020; Chuang et al., 2022), which show superior performance compared with standard supervised ImageNet models for some computer vision benchmarks. Conversely, medical images acquired using specific imaging protocols exhibit consistent anatomical structures (e.g., chest anatomy in FIG. 2), with clinically relevant information dispersed over the entire image (Haghighi et al., 2021). Particularly, high-level structural information (i.e., anatomical structures and their relative spatial orientations) is essential for the identification of both normal anatomy and various disorders. Also, medical tasks demand heightened attention to fine-grained details within images, as the identification of diseases, organ delineation, and lesion isolation depend on subtle, local variations in texture (Hosseinzadeh Taher et al., 2021). Thus, medical imaging recognition tasks rely heavily on the integration of fine-grained discriminative features extracted from the entire image. As a result, for medical vision tasks, restorative-based learning is preferred, as evidenced by the superior performance of restorative SSL approaches (Zhou et al., 2021b; Chen et al., 2019; Haghighi et al., 2021; Tao et al., 2020; Zhou et al., 2021a; Haghighi et al., 2020; Hosseinzadeh Taher et al., 2022) compared with their discriminative counterparts (Azizi et al., 2021; Zhou et al., 2020; Kaku et al., 2021) for various medical vision benchmarks.

Accordingly, systematic analysis has disclosed: (1) discriminative learning excels in capturing high-level (global) discriminative features; (2) restorative learning is optimal for conserving fine-grained details embedded in local image regions; and (3) adversarial learning consolidates restoration by conserving more fine-grained details. More importantly, a new and intriguing insight into trio of discriminative, restorative, and adversarial learning was acquired to excavate effective features required for medical recognition tasks—both high-level anatomical representations and fine-grained discriminative cues embedded in the local parts of medical images.

Based on the insights above, a novel self-supervised learning framework was developed, named DiRA, by uniting discriminative learning, restorative learning, and adversarial learning in a unified manner to glean complementary visual information from unlabeled medical images. As illustrated in FIG. 1, despite the critical contributions of discriminative, restorative, and adversarial learning to SSL performance, both individually and in pairs, existing efforts have not exploited the simultaneous utilization of all three learning ingredients in an SSL schema. In contrast, DiRA, a novel SSL framework, unites discriminative, restorative, and adversarial learning in a unified manner to collaboratively glean complementary visual information from unlabeled data for fine-grained semantic representation learning.

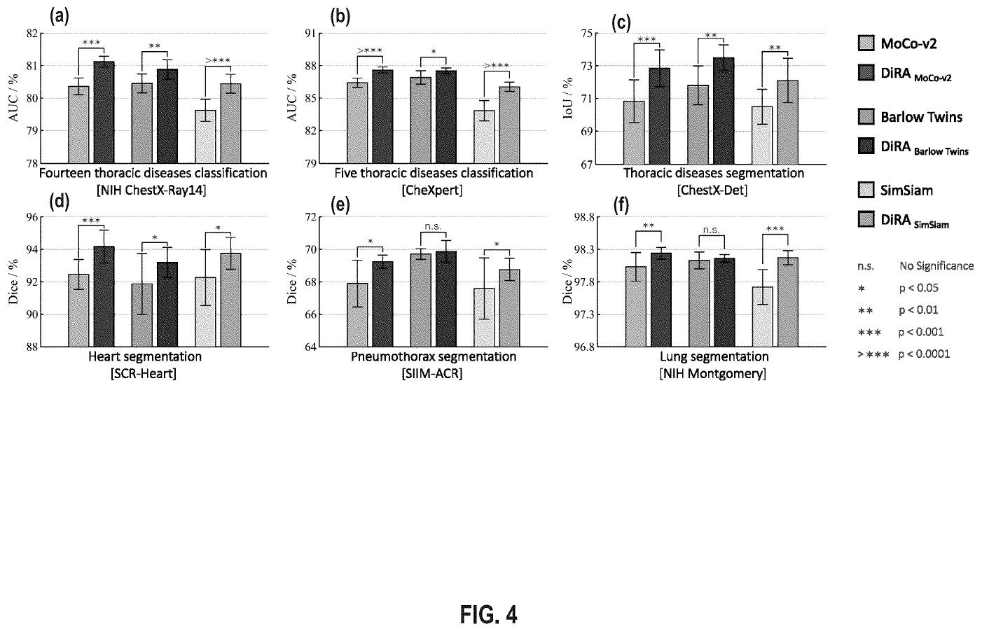

Extensive experiments demonstrate that (1) DiRA encourages collaborative learning among three learning components, resulting in more generalizable representation across distribution shifts, organs, diseases, and modalities. For example, FIG. 4 shows comparison with discriminative learning methods: DiRA was applied to three representative self-supervised methods with diverse discrimination objectives: MoCo-v2, Barlow Twins, and SimSiam. DiRA empowers these discriminative methods to retain more fine-grained representations, resulting in significant (p<0.05) performance boosts in six downstream tasks.

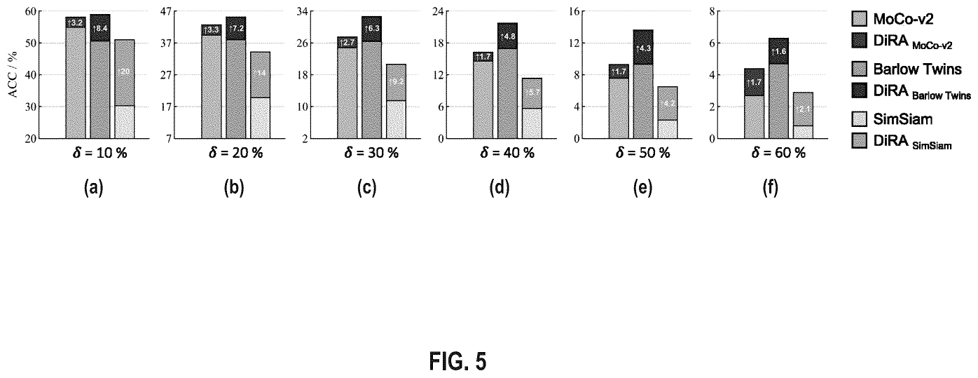

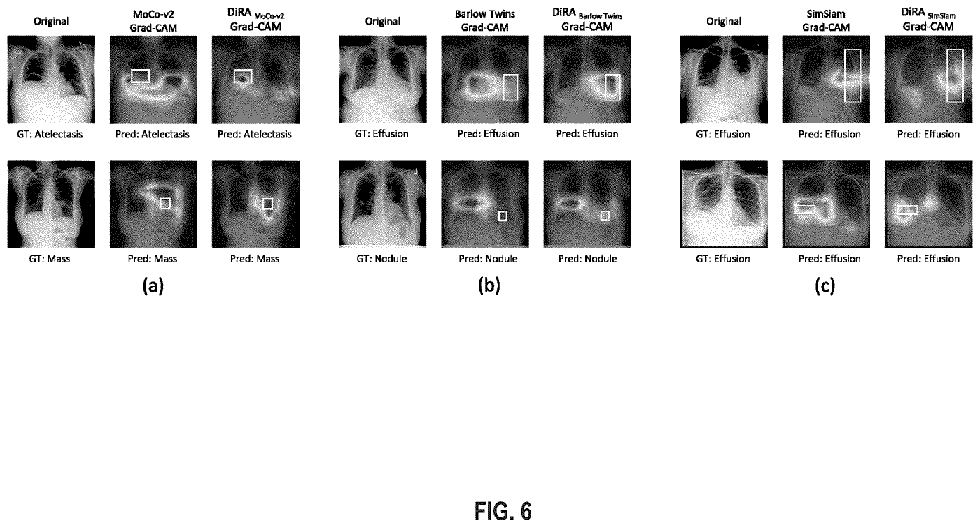

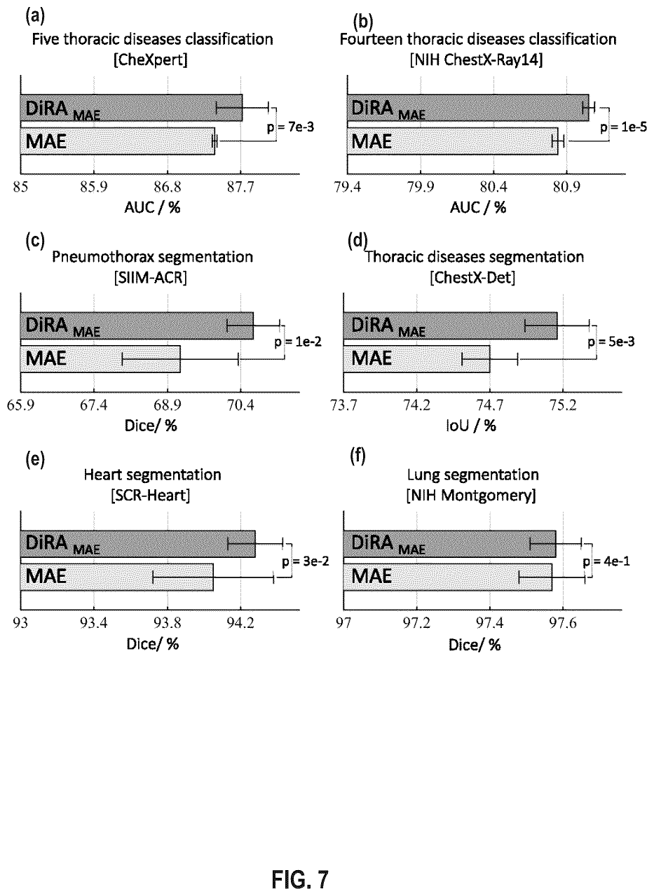

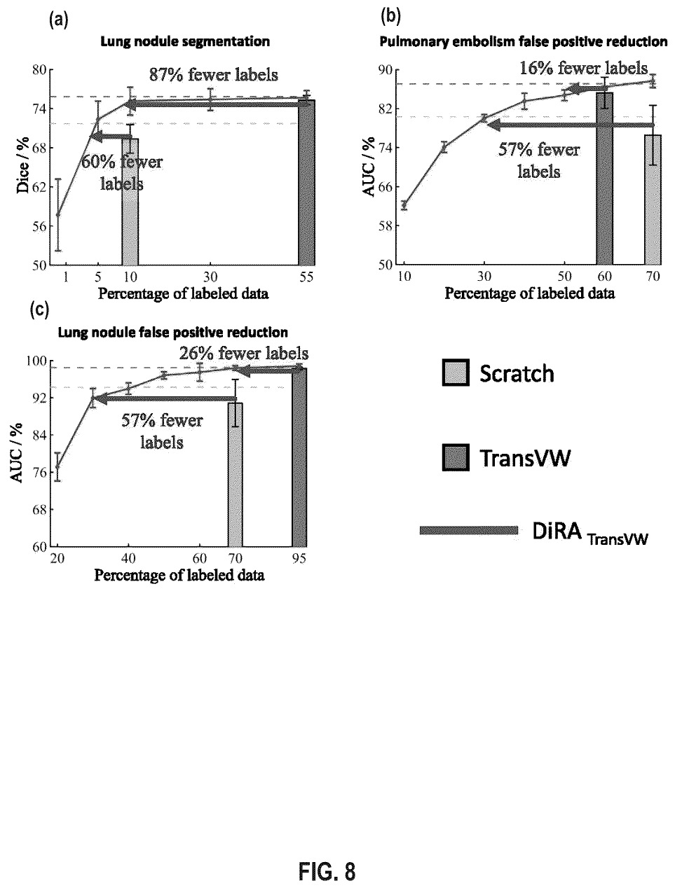

(2) DiRA also outperforms fully-supervised baselines models and increases robustness in small data regimes, thereby reducing annotation costs in medical imaging (see Tables 2 and 3); (3) DiRA learns fine-grained representations, facilitating more accurate lesion localization with only image-level annotations (see FIGS. 5 and 6); (4) DiRA provides highly reusable low/mid-level features, resulting in greater transferability to different medical tasks (see Table 4); and (5) DiRA enhances restorative-based approaches, showing that DiRA is a general framework for united representation learning (see FIG. 7, Table 5, Table 6, and FIG. 8).

DiRA provides at least the following contributions:
The insights gained into the synergy of discriminative, restorative, and adversarial learning in a ternary setup, realizing a new paradigm of collaborative learning for SSL.
The first self-supervised learning framework that seamlessly unites discriminative, restorative, and adversarial learning in a unified manner.
A thorough and insightful set of experiments that demonstrate not only DiRA's generalizability but also its potential to take a fundamental step towards developing universal representations for medical imaging.

### 2. DIRA FRAMEWORK

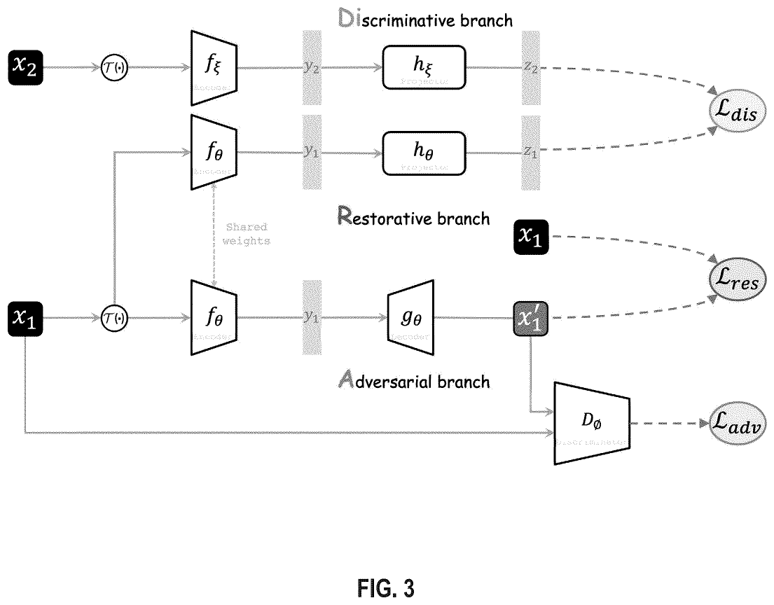

DiRA is a general self-supervised learning framework that learns comprehensive information from unlabeled medical images. As illustrated in FIG. 3, DiRA consists of three key learning components: (1) Discrimination (Di) which learns high-level discriminative representations, (2) Restoration (R) which forces the model to conserve fine-grained information about the image by focusing on more localized visual patterns, and (3) Adversary (A) which further improves feature learning through the restoration component. By integrating these components into a unified framework, DiRA captures complementary information from images, resulting in more pronounced representations for various downstream tasks.

In general, DiRA, aims to learn generalizable image representations by uniting three learning components: discriminative, restorative, and adversary. Given two input patches x₁ and x₂ of the same class/instance, random data augmentations T(·) are applied on them, and give them as the input to the discriminative and restorative branches. The discriminative branch consists of twin encoders f_{θ} and f_{ξ}, and projectors h_{θ} and h_{ξ}, and encodes the input samples to high-level embeddings vectors. The discriminative branch is trained to maximize the agreement between the (high-level) embedding vectors of the samples from the same (pseudo) class. The restorative branch consists of an encoder f_{θ} and decoder g_{θ}, and restores the original input samples from their distorted ones. The restorative branch is trained to maximize the (pixel-level) agreement between original samples and their restored versions. The adversarial branch consists of a discriminator network D_{ø}, and receives both the predictions of restorative branch and the original input samples as the input. The adversarial branch engages in a minimax game with the restorative branch; the adversarial branch attempts to distinguish between the original and restored samples, while the restorative branch attempts to confuse the adversarial discriminator by generating samples that are indistinguishable from real ones.

In the following sections, each component of DiRA is explained by abstracting a common paradigm and then detailing the joint training objective.

(1) Discriminative learning—encoding high-level discriminative representations. The discriminative learning component aims to model the similarities among instances of the same (pseudo) class in the embedding space. To do so, this component employs a discriminative objective function to learn global image representations that discriminate instances of different (pseudo) classes. As illustrated in FIG. 3, the discriminative learning branch is comprised of (1) two twin encoders f_{θ} and f_{ξ}, and (2) two projection heads h_{θ} and h_{ξ}. In this setting, f_{θ} is a regular encoder updated by back-propagation, while f_{ξ} can share weights with f_{θ} (Zbontar et al., 2021; Chen and He, 2021; Haghighi et al., 2021) or can be a momentum encoder (He et al., 2020; Grill et al., 2020) updated by an exponential moving average (EMA) of the parameters in f_{θ}. Given two input patches x₁ and x₁, which represent samples of the same (pseudo) class, we first apply an augmentation function [symbol] (·) on them. The two augmented patches are fed to encoders f_{θ} and f_{ξ}, producing their latent representations y₁=f_{θ}( [symbol] (x₁)) and y₂=f_{ξ}( [symbol] (x₂)). The latent representations are then mapped by projection heads h_{θ} and h_{ξ} onto the embeddings z₁=h_{θ}(y₁) and z₂=h_{ξ}(y₂). The discriminative branch is trained by maximizing the similarity between the embedding vectors of two samples that belong to the same (pseudo) class:

$${ℒ}_{dis} = ℓ ( {z}_{1} , {z}_{2} ) \qquad ( 1 )$$

where [symbol] (z₁, z₂) is the similarity/distance function that measures the agreement between z₁ and z₂. Discriminative learning yields a pre-trained encoder referred to as f_{Di}. DiRA is a general framework that allows various choices of discrimination tasks without any constraint. Therefore, the definition of a class can vary from treating each individual image as a separate class (instance-wise discrimination) to clustering images based on a similarity metric (cluster-wise discrimination). Accordingly, x₁ and x₂ can be two views of the same image or two samples belonging to the same cluster. Depending on the choice of the discrimination task, the instantiation of [symbol] _{dis} can be cross-entropy (Haghighi et al., 2021; Gidaris et al., 2018; Noroozi and Favaro, 2016; Zhuang et al., 2019), contrastive (He et al., 2020; Chen et al., 2020b; Azizi et al., 2021; Caron et al., 2021a), information maximization (Zbontar et al., 2021; Ermolov et al., 2021), distillation (Chen and He, 2021), etc.

(2) Restorative learning—conserving fine-grained visual details embedded in local image regions. The restorative learning component aims to improve discriminative learning by capturing fine-grained anatomical information—the subtle diversity of intensity, shape, boundary, and texture presented in different samples. To do so, this component employs a reconstruction objective function to capture local image representations. As illustrated in FIG. 3, the restorative learning branch is comprised of an encoder f_{θ} and decoder g_{θ}, with the encoder f_{θ} being shared with the discrimination branch. Given an input sample x₁ distorted by [symbol] , the f_{θ} maps the input sample to a latent representation and g_{θ} reconstructs the original sample from the latent representation, i.e., f_{θ}, g_{θ}:(x, [symbol] ) [symbol] x. The restoration branch is trained by minimizing the pixel-wise distance between the original sample and its reconstructed counterpart:

$${ℒ}_{res} = 𝔼 [ dist ( {x}_{1} , {x}_{1}^{′} ) ] \qquad ( 2 )$$

where x′₁=g_{θ}(f_{θ}( [symbol] (x₁))) denotes the reconstructed sample. dist(x₁,x′₁) presents the distance function that measures similarity between x₁ and x′₁ at a pixel-level, such as L₁ or L₂. The incremental addition of restorative learning to discriminative learning results in the pre-trained encoder and decoder being referred to as f_{Di(Di+R)} and g_{(Di+R)}, respectively.

(3) Adversarial learning—consolidating restorative learning by conserving more fine-grained details. The adversarial learning component aims to reinforce restorative learning by encouraging the reconstructed images to be indistinguishable from the original input images. To do so, this component exploits an adversarial objective function to capture more informative details from images so that the restorative branch can reproduce the original images more effectively. As illustrated in FIG. 3, we adapt the Generative Adversarial Network (GAN) paradigm (Cao et al., 2020; Parmar et al., 2021) in adversarial learning component by modeling the generator with the restorative component (i.e. f_{θ} and decoder g_{θ}) and introducing an adversarial discriminator D_{ϕ}. The learning procedure involves a two-player minimax game where the adversarial discriminator D_{ϕ} receives both the predictions of restorative branch and the original input samples, and attempts to distinguish between them. On the other hand, the restorative branch attempts to confuse the adversarial discriminator by generating samples that are indistinguishable from real ones. The restoration branch and the adversarial discriminator D_{ϕ} are optimized jointly with an adversarial loss:

$${ℒ}_{adv} = 𝔼 [ log {D}_{ϕ} ( {x}_{1} ) + log ( 1 - {D}_{ϕ} ( {x}_{1}^{′} ) ) ] \qquad ( 3 )$$

where x′₁=g₇₄ (f_{θ}( [symbol] (x₁))) denotes the reconstructed sample. The incremental addition of adversarial learning to discriminative and restorative learning results in the pretrained encoder f_{Di(Di+R)(Di+R+A)} and decoder g_{(Di+R)(Di+R+A)}. The pretrained encoder and encoder-decoder, referred to as DiRA models, are used for downstream classification and segmentation tasks, respectively.

To enable end-to-end representation learning from multiple sources of information and yield more powerful models for diverse medical tasks, DiRA integrates discriminative, restorative, and adversarial learning and jointly trained them with one single objective function:

$$ℒ = {λ}_{dis} * {ℒ}_{dis} + {λ}_{res} * {ℒ}_{res} + {λ}_{adv} * {ℒ}_{adv} \qquad ( 4 )$$

where λ_{dis}, λᵣₑₛ, and λ_{adv} are coefficients that balance the weights of different loss terms. Our unified training scheme enables DiRA to learn representations that conserve fine-grained information from images while being discriminative among different (pseudo) classes. Specifically, [symbol] _{dis} encourages the model to capture high-level discriminative features. Moreover, [symbol] ᵣₑₛ empowers discriminative learning by encoding finer and more discriminative information from the local context of images. Finally, [symbol] _{adv} elevates restorative learning through capturing more informative local features.

### 3. EXPERIMENTS

### 3.1. Pretraining DiRA

DiRA is a general self-supervised learning framework that is compatible with existing self-supervised discriminative and restorative methods, regardless of their objective functions. To evaluate the effectiveness of our framework, we adapt recent 2D and 3D self-supervised approaches into DiRA, detailed in the following sections. The resulting pretrained models with DiRA are denoted as DiRA subscripted by the original method name. DiRA models are pretrained solely on unlabeled images. Nevertheless, to prevent any test-case leakage from pretext tasks to downstream tasks, we exclude any images that will be used for testing in downstream tasks from pretraining stage.

### 3.1.1. 2D Pretraining Settings

We apply DiRA to four popular convolutional neural network (ConvNet)- and vision-transformer-based SSL methods with diverse training objectives, including MoCo-v2 (Chen et al., 2020a), Barlow Twins (Zbontar et al., 2021), SimSiam (Chen and He, 2021), and MAE (He et al., 2022). We adapt discriminative SSL approaches, including MoCo-v2, Barlow Twins, and SimSiam, in DiRA by adding restorative and adversarial learning to their training schemes. For each of these three discrimination tasks (Chen et al., 2020a; Zbontar et al., 2021; Chen and He, 2021), we adhere to the original methods in the formulation of discriminative learning loss ( [symbol] _{dis}), projection head architecture, and hyperparameter settings. We adopt mean square error (MSE) between the input and the reconstructed images as the restorative learning loss ( [symbol] ᵣₑₛ). For all discriminative methods, we employ a 2D U-Net (Ronneberger et al., 2015) with a standard ResNet-50 (He et al., 2016) backbone as the f_{θ} and g_{θ}, and optimize them following the setups in Chen et al. (2020a), Zbontar et al. (2021), Chen and He (2021). We adapt the restorative SSL approach (i.e. MAE) in DiRA by adding discriminative and adversarial learning to its training objective. Specifically, we use a ViT-S/16 encoder and follow (Xiao et al., 2023) in the restoration task settings, decoder architecture, and optimization parameters. We utilize contrastive loss (Chen et al., 2021c; Mishra et al., 2022) as the discriminative learning loss ( [symbol] _{dis}). For both discriminative and restorative SSL methods, we use a lightweight network consisting of four convolutional layers with the kernel size of 3×3 (Pathak et al., 2016) as the discriminator network (D_{ϕ}), and optimize it using the Adam optimizer with a learning rate of 2e-4 and (β₁, β₂)=(0.5,0.999). All DIRA models are pretrained from scratch on the training set of ChestX-ray14 (Wang et al., 2017) dataset. We use a batch size of 256 distributed across 4 Nvidia V100 GPUS. For discriminative SSL approaches, λᵣₑₛ, λ_{adv}, λ_{dis} are empirically set to 10, 0.001, and 1, respectively. For the restorative SSL approach, λᵣₑₛ, λ_{adv}, λ_{dis}_{^{s}} are set to 0.67, 0.001, and 0.03, respectively. Input images are first randomly cropped and resized to 224×224. The image augmentation function ( [symbol] (·)) includes random horizontal flipping, color jittering, and Gaussian blurring. Additionally, we apply cutout (DeVries and Taylor, 2017; Pathak et al., 2016) and shuffling (Chen et al., 2019) to enrich the restoration task for the three discriminative SSL approaches. More details are provided in the Appendix B.

### 3.1.2. 3D Pretraining Settings

We apply DiRA to TransVW (Haghighi et al., 2021) as a representative method for 3D self-supervised learning in medical imaging. We adapt TransVW in DiRA by adding adversarial learning to its training scheme. To ensure fair comparisons, we follow the publicly available TransVW in the formulation of visual words discrimination and restoration tasks, as well as hyperparameter settings. In particular, we employ a 3D U-Net (Ellis and Aizenberg, 2021) as the encoder-decoder network, with a classification head appended at the end of the encoder. The adversarial discriminator D_{ϕ} includes four convolutional blocks with the kernel size 3×3×3. λᵣₑₛ, λ_{adv}, λ_{dis} are empirically set to 100, 1, and 1, respectively. Moreover, similar to TransVW, DiRA models are pretrained from scratch using 623 chest CT scans in the LUNA16 (Setio et al., 2017) dataset. f_{θ}, g_{θ}, and D_{ϕ} are optimized for 200 epochs using Adam with a learning rate of 1e-3 and batch size of 8. More details are in the Appendix B.

### 3.2. Fine-Tuning DiRA

### 3.2.1. Downstream Tasks and Datasets

We evaluate the effectiveness of DiRA's representations in transfer learning to a diverse suite of 12 common but challenging medical imaging tasks on eleven public datasets. The downstream tasks include classification, segmentation, and weakly-supervised disease localization in 2D and 3D images. As summarized in Table 1 and detailed below, these downstream tasks encompass various label structures (multi-label classification, pixel-level disease/organ segmentation, and weakly-supervised disease localization), diseases (brain tumors and thoracic diseases, such as lung nodules, pulmonary embolism, and pneumothorax), organs (lung, liver, brain, and heart), and modalities (X-ray, CT, and MRI). Additionally, these tasks present several typical challenges encountered in medical imaging, such as class imbalance, limited data, and small scan areas for the pathology of interest. We adopt the official data splits of these datasets whenever available; otherwise, we randomly split the data into 80% for training and 20% for testing.

ChestX-ray14 (Wang et al., 2017) is a large-scale open source dataset of de-identified chest radiographs. The dataset includes 112K chest radiographs taken from 30K unique patients. The ground truth consists of a label space of 14 thorax diseases. We use the official patient-wise split released with the dataset, including 86K training images and 25K testing images. The models are trained to predict 14 pathologies in a multi-label classification setting. The mean AUC score over 14 diseases is used to evaluate the classification performance. In addition to image-level labels, ChestX-ray14 provides bounding box annotations for approximately 1,000 test images. Of this set of images, bounding box annotations are available for 8 out of 14 thorax diseases. During testing, we use bounding box annotations to assess the accuracy of pathology localization in a weakly-supervised setting. The mean accuracy over 8 diseases is used to evaluate the localization performance.

CheXpert (Irvin et al., 2019) is a hospital-scale publicly available dataset with 224K chest radiographs taken from 65K unique patients. We use the official data split released with the dataset, including 224K training and 234 test images. The ground truth for the training set includes 14 thoracic pathologies that were retrieved automatically from radiology reports. The testing set is labeled manually by board-certified radiologists for 5 selected thoracic pathologies—cardiomegaly, edema, consolidation, atelectasis, and pleural effusion. The models are trained to predict five pathologies in a multi-label classification setting. The mean AUC score over 5 diseases is used to evaluate the classification performance.

TABLE 1
Downstream tasks for transfer learning: we evaluate the learned representations by our SSL framework
in 12 medical imaging applications, including classification, segmentation, and weakly-supervised
localization on 2D and 3D images. To assess the generalization ability of our DiRA, we select
a diverse set of applications ranging from the tasks on the same dataset as pretraining to the
tasks with substantial shifts in data distribution and the disease/organ of interest.

| Dataset | Downstream task | Object | Modality |
|---|---|---|---|
| ChestX-ray14 (Wang et al., 2017) | Fourteen thoracic | Thoracic Diseases | X-ray |
| | diseases classification | | |
| CheXpert (Irvin et al., 2019) | Five thoracic | Thoracic Diseases | X-ray |
| | diseases classification | | |
| SIIM-ACR (Zawacki et al., 2019) | Pneumothorax segmentation | Pneumothorax | X-ray |
| NIH Montgomery (Jaeger et al., 2014) | Lung segmentation | Lung | X-ray |
| SCR-Heart (van Ginneken et al., 2006) | Heart segmentation | Heart | X-ray |
| ChestX-Det (Lian et al., 2021) | Thoracic diseases segmentation | Thoracic Diseases | X-ray |
| ChestX-ray14 (Wang et al., 2017) | Weakly-supervised | Thoracic Diseases | X-ray |
| | disease localization | | |
| LUNA16 (Setio et al., 2017) | Lung nodule false | Lung Nodule | CT |
| | positive reduction | | |
| LIDC-IDRI (Armato et al., 2011) | Lung nodule segmentation | Lung Nodule | CT |
| PE-CAD (Tajbakhsh et al., 2015) | Pulmonary embolism (PE) | Pulmonary Embolism | CT |
| | false positive reduction | | |
| LiTS-2017 (Bilic et al., 2019) | Liver segmentation | Liver | CT |
| BraTS2018 (Bakas et al., 2018) | Brain tumor segmentation | Brain Tumor | MRI Flair |

SIIM-ACR (Zawacki et al., 2019) dataset is provided by the Society for Imaging Informatics in Medicine (SIIM) and American College of Radiology, including 10K chest radiographs and pixel-wise segmentation mask for pneumothorax disease. We randomly divided the dataset into training (80%) and testing (20%). The models are trained to segment pneumothorax in images (if present). The mean Dice score is used to evaluate the segmentation performances.

NIH Montgomery (Jaeger et al., 2014) dataset is provided by the Montgomery County's Tuberculosis screening program, including 138 chest radiographs. There are 80 normal cases and 58 cases with tuberculosis (TB) indications in this dataset. Ground truth segmentation masks for left and right lungs are provided. We randomly divided the dataset into a training set (80%) and a test set (20%). The models are trained to segment left and right lungs in chest scans. The mean Dice score is employed to evaluate the segmentation performances.

SCR-Heart (van Ginneken et al., 2006) dataset includes 247 chest radiographs from JSRT database (Shiraishi et al., 2000) along with segmentation masks for the heart. The data has been subdivided into two folds with 124 and 123 images. We follow the official split of the dataset, using fold1 for training (124 images) and fold2 for testing (123 images). The mean Dice score is used to evaluate the heart segmentation performances.

ChestX-Det (Lian et al., 2021) dataset consists of 3,578 images selected from ChestX-ray14 dataset along with segmentation masks for 13 thoracic diseases, including atelectasis, calcification, cardiomegaly, consolidation, diffuse nodule, effusion, emphysema, fibrosis, fracture, mass, nodule, pleural thickening, and pneumothorax. The image annotations are provided by 3 board-certified radiologists. The downstream task is pixel-wise segmentation of lesions in images. We adhere to the official dataset split, using 3,025 images for training and 553 images for testing. The mean IoU score is utilized to evaluate the segmentation performance.

LUNA16 (Setio et al., 2017) dataset consists of 888 lung CT scans with a slice thickness of less than 2.5 mm. The data were divided into training (445 cases), validation (178 cases), and test (265 cases) sets. The dataset provides a set of 5M candidate locations for lung nodule, each is labeled as true positive or false positive. The models are trained to classify lung nodule candidates into true positives and false positives in a binary classification setting. The Area Under the Curve (AUC) score is used to evaluate the classification accuracy.

LIDC-IDRI (Armato et al., 2011) dataset includes 1,018 chest CT scans with annotated lung nodules. The dataset is divided into training (510 cases), validation (100 cases), and test (408 cases) sets. We preprocessed the data by re-sampling the 3D volumes to 1-1-1 spacing and then extracting a 64×64×32 crop around each nodule. The models are trained to segment long nodules in these 3D crops. The Intersection over Union (IoU) metric is used to measure the segmentation accuracy.

PE-CAD (Tajbakhsh et al., 2015) dataset includes 121 computed tomography pulmonary angiography (CTPA) scans with a total of 326 pulmonary embolism (PE). The dataset provides a set of candidate locations for PE and is divided at the patient-level into training and test sets. Training set contains 434 true positive and 3,406 false positive PE candidates. Test set contains 253 true positive and 2,162 false positive PE candidates. We pre-processed the 3D scans as suggested in Zhou et al. (2021b). The 3D models are trained to classify PE candidates into true positives and false positives in a binary classification setting. The Area Under the Curve (AUC) score at the candidate-level is used to evaluate the classification accuracy.

LiTS (Bilic et al., 2019) dataset is provided by MICCAI 2017 LiTS Challenge, including 130 CT scans with expert ground-truth segmentation masks for both liver and liver tumors. We divide dataset into training (100 patients), validation (15 patients), and test (15 patients) sets. The models are trained to segment liver in 3D scans. The Intersection over Union (IoU) metric is used to measure the segmentation accuracy.

BraTS (Bakas et al., 2018) dataset includes brain MRI scans of 285 patients (210 HGG and 75 LGG) and segmentation masks for necrotic and non-enhancing tumor core, peritumoral edema, GD-enhancing tumor, and background. For each patient, four different MR volumes are available: native T1-weighted (T1), post-contrast T1-weighted (T1Gd), T2-weighted (T2), and T2 fluid attenuated inversion recovery (FLAIR). We divide dataset at patient-level into training (190 patients) and testing (95 patients) sets. The models are trained to segment brain tumors (background as negatives class and tumor sub-regions as positive class). The mean Intersection over Union (IoU) metric is used to measure the segmentation accuracy.

### 3.2.2. Evaluations

For transfer learning to the classification tasks, we utilize the pre-trained encoder of DiRA models (f_{θ}) and append a task-specific classification head to them. For transferring ConvNet-based DiRA models to the segmentation tasks, we utilize the pretrained encoder and decoder of DiRA models (f_{θ} and g_{θ}) and replace the last layer of the decoder with a 1×1 and 1×1×1 convolutional layer for 2D and 3D, respectively. For transferring ViT-based DiRA model (i.e. DiRA_{MAE}) to the segmentation tasks, we use the pretrained encoder and follow (Chen et al., 2021a) for the decoder architecture. We evaluate the generalization of DiRA's representations by fine-tuning all parameters in downstream models. We use the AUC (area under the ROC curve), and the IoU (Intersection over Union) and Dice coefficient to evaluate the accuracy of classification and segmentation performances, respectively. We strive to optimize each downstream task with the best performing hyperparameters. For 2D downstream tasks, we resize the images to 224×224, and apply standard data augmentation techniques specific to each task. For classification tasks, we employ random cropping and resizing, horizontal flipping, and rotating. For segmentation tasks, we utilize random brightness contrast, random gamma, optical distortion, elastic transformation, and grid distortion. For 3D target tasks, we follow (Zhou et al., 2021b; Haghighi et al., 2021) in using random flipping, transposing, rotating, and adding Gaussian noise as data augmentations. In all 2D and 3D downstream tasks with ConvNet architecture, we use Adam optimizer with β₁=0.9, β₂=0.999. For downstream task with ViT architecture, following (Xiao et al., 2023; Chen et al., 2021a), we use AdamW optimizer for classification and SGD optimizer for segmentation tasks. We employ early-stop mechanism using the 10% of the training data as the validation set to avoid over-fitting. For 2D classification tasks with ConvNet backbone on ChestX-ray14 and CheXpert datasets, we use a learning rate 2e-4 and ReduceLROnPlateau as the learning rate decay scheduler. For 2D classification tasks with ViT backbone, we closely follow (Xiao et al., 2023) in training settings. For 2D segmentation tasks on SCR-Heart, ChestX-Det, SIIM-ACR, and Montgomery, we use cosine learning rate decay scheduler. For 2D segmentation tasks with ConvNet backbone, we use a learning rate 1e-3. For 2D segmentation tasks with ViT backbone, we use a learning rate 1e-2 in SCR-Heart and Montgomery, and 1e-3 in SIIM-ACR and ChestX-Det datasets. For all 3D downstream tasks, we use ReduceLROnPlateau as the learning rate decay scheduler. For downstream tasks on LUNA16, PE-CAD, LIDC, and LiTS, we use a learning rate 1e-2, whereas we employ a learning rate of 1e-3 for BraTS. We run each method 10 times on each downstream task and report the average, standard deviation, and statistical analysis based on an independent two-sample t-test.

### 4. RESULTS

This section presents the cornerstones of our results, showcasing not only the properties of our self-supervised framework but also its generalizability across a myriad of downstream tasks. Through a series of experiments, we demonstrate that DiRA (1) fosters collaborative learning among three learning components, capturing more diverse visual representations that exhibit superior generalization capabilities across different tasks compared with both self-supervised and fully-supervised baselines (Sections 4.1 and 4.4); (2) provides more robust models for downstream tasks with limited data, offering an annotation-efficient solution for medical imaging (Section 4.2); (3) learns fine-grained discriminative features, facilitating more precise disease localization with only image-level annotations (Section 4.3); (4) enhances the reusability of low/mid-level features, resulting in greater transferability to different medical tasks (Section 4.5); and (5) elevates restorative SSL approaches, demonstrating its versatility as a general framework for united representation learning (Section 4.6).

### 4.1. DiRA Synergizes Discriminative, Restorative, and Adversarial Learning

Experimental setup. We assess the flexibility and efficacy of our proposed self-supervised framework by applying it to three recent popular self-supervised methods with diverse discrimination formulations, including (1) MoCo-v2 (Chen et al., 2020a)—representative of contrastive learning SSL methods, (2) Barlow Twins (Zbontar et al., 2021)—representative of information maximization SSL methods, and (3) SimSiam (Chen and He, 2021)—representative of self-distillation SSL methods. To evaluate the quality of the learned representations by our DiRA and ascertain the generality of our findings, we fine-tune all models on a broad range of six downstream tasks, covering classification (ChestX-ray14 and CheXpert) and segmentation (SCR-Heart, ChestX-Det, SIIM-ACR, and NIH Montgomery).

Results. FIG. 4 showcases the enhancements achieved by integrating DiRA with MoCo-v2, Barlow Twins, and SimSiam across all downstream tasks, including (1) ChestX-ray14, (2) CheXpert, (3) SCR-Heart, (4) ChestX-Det, (5) SIIM-ACR, and (6) NIH Montgomery. Compared with the original methods, DiRA_{MoCo-v2} yields increased performance by 0.8%, 1.2%, 1.7%, 2.0%, 1.4%, and 0.2%, respectively. Similarly, DiRA_{Barlow Twins} yields increased performance by 0.4%, 0.6%, 1.3%, 1.7%, 0.2%, and 0.03%. Finally, DiRA_{SimSiam} yields increased performance by 0.8%, 2.2%, 1.5%, 1.6%, 1.2%, and 0.5%. These results suggest that DiRA provides a comprehensive representation learning framework that encourages existing self-supervised discriminative approaches to preserve more fine-grained information from images, thereby enriching their visual representations and enabling more effective generalization to diverse medical tasks.

### 4.2. DiRA Improves Robustness to Small Data Regimes

Experimental setup. We investigate the robustness of representations learned with DiRA in small data regimes to determine if the learned representation can serve as a proper foundation for fine-tuning in applications with a dearth of annotated data. We randomly sample different fractions (1%, 5%, 10%, and 25%) of training data from the ChestX-ray14, CheXpert, Montgomery, and ChestX-Det datasets, and fine-tune the self-supervised pretrained models on these training data subsets.

TABLE 2
Fine-tuning the SSL pretrained models using different downstream label fractions: DiRA models combat overfitting
in low data regimes and provide stronger representations for downstream tasks with limited annotated data.
For each application, the average performance over multiple runs has been reported. The green numbers show
the improvements of DiRA models compared with the underlying discriminative method in each data portion.

| | ChestX-ray14 [AUC (%)] | | | | CheXpert [AUC (%)] | | | | Montgomery [Dice (%)] | |
|---|---|---|---|---|---|---|---|---|---|---|
| | Label fraction | | | | Label fraction | | | | Label fraction | |
| Method | 1% | 5% | 10% | 25% | 1% | 5% | 10% | 25% | 1% | 5% |
| MoCo-v2 | 52.99 | 59.29 | 67.17 | 74.89 | 76.87 | 78.37 | 80.24 | 81.70 | 63.69 | 72.95 |
| DiRA_{MoCo-v2} | 59.39 | 65.64 | 73.45 | 77.55 | 78.43 | 81.54 | 85.43 | 87.12 | 72.53 | 81.81 |
| Performance boost | (+6.40) | (+6.35) | (+6.28) | (+2.66) | (+1.56) | (+3.17) | (+5.19) | (+5.42) | (+8.84) | (+8.86) |
| Barlow Twins | 62.43 | 67.77 | 73.14 | 76.23 | 82.85 | 83.00 | 83.19 | 83.74 | 86.79 | 88.21 |
| DiRA_{Barlow Twins} | 62.51 | 68.36 | 73.30 | 77.18 | 83.12 | 83.30 | 83.52 | 84.20 | 87.25 | 90.20 |
| Performance boost | (+0.08) | (+0.59) | (+0.16) | (+0.95) | (+0.27) | (+0.30) | (+0.33) | (+0.46) | (+0.46) | (+1.99) |
| SimSiam | 51.07 | 58.28 | 67.03 | 73.05 | 65.39 | 71.39 | 79.15 | 80.05 | 48.20 | 55.62 |
| DiRA_{SimSiam} | 53.42 | 59.95 | 68.10 | 74.38 | 70.46 | 74.46 | 79.47 | 81.03 | 61.86 | 68.31 |
| Performance boost | (+2.35) | (+1.67) | (+1.07) | (+1.33) | (+5.07) | (+3.07) | (+0.32) | (+0.98) | (+13.66) | (+12.69) |

| | Montgomery [Dice (%)] | | ChestX-Det [IoU (%)] | | | |
|---|---|---|---|---|---|---|
| | Label fraction | | Label fraction | | | |
| Method | 10% | 25% | 1% | 5% | 10% | 25% |
| MoCo-v2 | 82.02 | 96.44 | 26.02 | 44.86 | 61.60 | 65.65 |
| DiRA_{MoCo-v2} | 92.16 | 97.06 | 38.57 | 50.86 | 66.22 | 69.83 |
| Performance boost | (+10.14) | (+0.62) | (+12.55) | (+6.00) | (+4.62) | (+4.18) |
| Barlow Twins | 91.45 | 97.49 | 50.33 | 57.26 | 65.90 | 69.32 |
| DiRA_{Barlow Twins} | 94.50 | 97.62 | 50.48 | 57.79 | 66.12 | 69.62 |
| Performance boost | (+3.05) | (+0.13) | (+0.15) | (+0.53) | (+0.22) | (+0.30) |
| SimSiam | 81.12 | 94.86 | 35.92 | 47.40 | 60.07 | 66.09 |
| DiRA_{SimSiam} | 93.67 | 96.61 | 45.76 | 54.32 | 64.19 | 67.50 |
| Performance boost | (+12.55) | (+1.75) | (+9.84) | (+6.92) | (+4.12) | (+1.41) |

Results. As seen in Table 2, DiRA models outperform their underlying original methods in all data subsets, i.e., 1%, 5%, 10%, and 25%, in ChestX-ray14, CheXpert, Montgomery, and ChestX-Det. In particular, the average of improvement for MoCo-v2 and SimSiam across all four downstream tasks in each underlying subset yielding: (1) 7.3% and 7.7% when using 1%, (2) 6.1% and 6.1% when using 5%, (3) 6.6% and 4.5% when using 10%, and (4) 3.2% and 1.4% when using 25%. Notably, in the 1% subset, DiRA models exhibit a substantial performance gain over MoCo-v2 and SimSiam, showcasing the potential of our framework in mitigating overfitting in extremely low data scenarios. While Barlow Twins exhibits greater resilience in low-data regimes compared with the two previous approaches, DiRA further enhances its performance by an average of 0.2%, 0.8%, 0.9%, and 0.5% across all four datasets when utilizing 1%, 5%, 10%, and 25% of the training data, respectively. In conclusion, our results in low-data regimes highlight the superiority of our framework in capturing more robust and transferable representations, paving the way for developing more accurate models for downstream tasks with limited labeled data and reducing annotation costs.

### 4.3. DiRA Enhances Weakly-Supervised Disease Localization

Experimental setup. We investigate the effectiveness of our DiRA framework in a weakly-supervised setting, comparing its applicability for localizing thoracic diseases to the underlying discriminative methods. To do so, we use the ChestX-ray14 dataset, which includes bounding box annotations for eight thoracic diseases in 983 images, as described in Wang et al. (2017). Bounding box annotations are only employed during the inference phase as ground truth to evaluate the disease localization accuracy. During the training phase, we initialize downstream models with SSL-pretrained models and fine-tune them using only image-level disease labels from ChestX-ray14 dataset. We then employ GradCAM (Selvaraju et al., 2017) to generate heatmaps, which identify the spatial location of specific thoracic diseases. For quantitative analyses, we follow the common protocol (Wang et al., 2017; Choe et al., 2020; Selvaraju et al., 2017) and first normalize each heatmap using min-max normalization; then, following (Wang et al., 2017), we binarize the heatmaps and generate bounding boxes around the isolated regions. We compute the intersection over union (IoU) between the computed and ground truth bounding boxes to assess localization accuracy. Following (Wang et al., 2017; Choe et al., 2020), a localization is considered correct when the bounding box prediction overlaps with the ground truth bounding box with IoU≥δ, where δ represents an overlap threshold. To provide a more comprehensive assessment, we investigate the localization accuracy under various values of 8, ranging from 10% to 60%. For each value of δ, we run each method at least five times and report the average accuracy across all runs.

Quantitative results. FIG. 5 depicts the mean localization accuracy over eight diseases for DiRA models and the underlying discriminative models. In particular, FIG. 5 shows weakly-supervised pathology localization accuracy under six IoU thresholds (δ): DiRA models provide stronger representations for pathology localization with only image-level annotations. For each method, the average performance over ten runs is reported. The white numbers show the improvement of DiRA models compared with the underlying discriminative method in each IoU threshold. The results demonstrate that DiRA models offer significant improvements in disease localization across all IoU thresholds compared with the original discriminative methods. Specifically, DiRA_{MoCo-v2}, DiRA_{Barlow Twins}, and DiRA_{SimSiam} exhibit average improvements of 2.4%, 5.4%, and 9.4% in disease localization across all IoU thresholds.

Qualitative results. FIG. 6 displays the visualization of Grad-CAMs for DiRA and the original discriminative methods. In particular, FIG. 6 shows visualization of Grad-CAM heatmaps for (a) MoCo-v2 vs. DiRA_{MoCo-v2}, (b) Barlow Twins vs. DiRA_{Barlow Twins}, and (c) SimSiam vs. DiRA_{SimSiam}: Transfer learning from DiRA models leads to improvements in weakly-supervised disease localization. While both DiRA and the underlying original models correctly predict the disease label in the test images, DiRA models capture the diseased regions more accurately than the baselines which either highlight larger regions of the image (e.g., (c), second row) or provide inaccurate localization without overlap with the ground truth (e.g., (b), second row). The white boxes depict the ground truth bounding box annotations.

As seen, the subject framework learns more fine-grained representations, leading to more precise disease localization. Specifically, the heatmaps provided by MoCo-v2, Barlow Twins, and SimSiam models exhibit high variability, whereas DiRA models consistently demonstrate more robust and accurate disease localization results compared with each respective original method. More visualizations are provided in Appendix A.

In conclusion, the quantitative and qualitative results indicate that our SSL framework yields a more fine-grained visual feature space, which captures essential cues for disease localization. Additionally, through the production of more interpretable activation maps, our DiRA framework demonstrates possible clinical potential for post-hoc interpretation by radiologists.

TABLE 3
Comparison with fully-supervised baselines: DiRA models provide superior or on-par performance compared with fully-supervised pretrained
models on ImageNet and ChestX-ray14 datasets in five downstream tasks. The best methods are bolded while the second best are underlined.

| | Pretraining | Classification | | Segmentation | | | |
|---|---|---|---|---|---|---|---|
| Method | Dataset | ChestX-ray14 | CheXpert | SIIM-ACR | SCR-Heart | Montgomery | ChestX-Det |
| Random Init. | — | 80.31 ± 0.10 | 86.62 ± 0.15 | 67.54 ± 0.60 | 88.87 ± 5.36 | 97.55 ± 0.36 | 71.22 ± 1.87 |
| Supervised | ImageNet | 81.70 ± 0.15 | 87.17 ± 0.22 | 67.93 ± 1.45 | 93.58 ± 1.03 | 98.19 ± 0.13 | 73.07 ± 0.69 |
| Supervised | ChestX-ray14 | N/A | 87.40 ± 0.26 | 68.92 ± 0.98 | 93.59 ± 1.22 | 98.16 ± 0.05 | 73.13 ± 0.72 |
| DiRA_{MoCo-v2} | ChestX-ray14 | 81.12 ± 0.17 | 87.59 ± 0.28 ‡ ‡ | 69.24 ± 0.41 ‡ † | 94.17 ± 1.01 † † | 98.24 ± 0.09 † ‡ | 72.84 ± 1.13 † † |
| DiRA_{Barlow Twins} | ChestX-ray14 | 80.88 ± 0.30 | 87.50 ± 0.27 ‡ † | 69.87 ± 0.68 ‡ ‡ | 93.19 ± 0.93 † † | 98.16 ± 0.06 † † | 73.49 ± 0.78 † † |
| DiRA_{SimSiam} | ChestX-ray14 | 80.44 ± 0.29 | 86.04 ± 0.43 | 68.76 ± 0.69 † † | 93.75 ± 0.98 † † | 98.17 ± 0.11 † † | 72.10 ± 1.36 † † |

‡ and ‡ denote the statistically significant (p < 0.05) improvement compared with the supervised ImageNet and ChestX-ray14 baselines, respectively, while † and † denote the statistically equivalent performances accordingly. For supervised ChestX-ray14 model, transfer learning to ChestX-ray14 is not applicable since pretraining and downstream tasks are the same, denoted by “N/A”. “Random Init.” denotes random initialization.

TABLE 4
Comparison of feature reuse between DiRA and the original discriminative SSL methods: each row presents
the CKA score for different model layers before and after fine-tuning in two downstream tasks, including
classification (ChestX-ray14) and segmentation (Montgomery). DiRA models provide more reusable low/mid
level features (layers 1-3) compared with the original discriminative approaches.

| | Pretraining | ChestX-ray14 | | | | | Montgomery | | | | |
|---|---|---|---|---|---|---|---|---|---|---|---|
| Method | dataset | Layer 1 | Layer 2 | Layer 3 | Layer 4 | Layer 5 | Layer 1 | Layer 2 | Layer 3 | Layer 4 | Layer 5 |
| MoCo-v2 | ChestX-ray14 | 0.995 | 0.622 | 0.625 | 0.500 | 0.409 | 0.995 | 0.618 | 0.695 | 0.546 | 0.339 |
| DiRA_{MoCo-V2} | ChestX-ray14 | 0.999 | 0.944 | 0.897 | 0.704 | 0.324 | 0.999 | 0.930 | 0.869 | 0.597 | 0.382 |
| Barlow Twins | ChestX-ray14 | 0.948 | 0.709 | 0.613 | 0.787 | 0.482 | 0.916 | 0.627 | 0.779 | 0.745 | 0.297 |
| DiRA_{Barlow Twins} | ChestX-ray14 | 0.996 | 0.922 | 0.718 | 0.794 | 0.450 | 0.992 | 0.914 | 0.874 | 0.704 | 0.395 |
| SimSiam | ChestX-ray14 | 0.977 | 0.629 | 0.677 | 0.683 | 0.480 | 0.978 | 0.527 | 0.577 | 0.532 | 0.402 |
| DiRA_{SimSiam} | ChestX-ray14 | 0.997 | 0.925 | 0.782 | 0.504 | 0.298 | 0.993 | 0.904 | 0.823 | 0.534 | 0.231 |

### 4.4. DiRA Outperforms Fully-Supervised Baselines

Experimental setup. We evaluate the transferability of DiRA models, which have been only pretrained on unlabeled images from ChestX-ray14 dataset, against two competitive fully-supervised pretrained models: (1) the supervised ImageNet model, which is the common transfer learning approach in medical imaging (Azizi et al., 2021; Hosseinzadeh Taher et al., 2021), and (2) a supervised model that has been pretrained on ChestX-ray14, which serves as an upper-bound in-domain transfer learning baseline. Both supervised baselines use the same encoder (ResNet-50) as DiRA models. We fine-tune all pre-trained models for six diverse applications, ranging from target tasks on the source dataset to tasks that involve substantial shifts in data distribution and the disease/object of interest.

Results. As shown in Table 3, DiRA models, pretrained with zero annotated data, yield higher or comparable performance compared with fully-supervised ImageNet and ChestX-ray14 models that have been pretrained using 1.3M and 86K labeled images, respectively. Particularly, DiRA_{MoCo-v2} outperforms both supervised baselines in CheX-pert, SCR-Heart, SIIM-ACR, and Montgomery. Also, DiRA_{Barlow Twins} shows superiority over supervised baselines in CheXpert, SIIM-ACR, and ChestX-Det. Finally, DiRA_{SimSiam} outperforms the supervised Im-ageNet model in SCR-Heart and SIIM-ACR, and the supervised ChestX-ray14 model in SCR-Heart and Montgomery. These results imply that our SSL framework can provide more versatile features than supervised pretraining, highlighting its potential for reducing future annotation costs in medical imaging.

### 4.5. DiRA Provides More Reusable Low/Mid-Level Features

Convolutional neural networks are widely recognized for their ability to construct feature hierarchies—lower layers of the network tend to learn more general, low/mid-level features, while higher layers are more likely to capture task-specific features (Neyshabur et al., 2020; Haghighi et al., 2021). The advantages of transfer learning are typically attributed to the reuse of pretrained low/mid-level features in downstream tasks (Asano et al., 2020; Zhao et al., 2021; Islam et al., 2021; Raghu et al., 2019). Higher feature reuse indicates that a self-supervised pretrained model captures more useful features, leading to higher performance in downstream tasks, especially those with limited annotated data (Kaku et al., 2021). Thereby, to further assess the quality of representations learned by our DiRA framework, we go beyond the transfer learning performance and conduct complementary feature reuse analyses.

Experimental setup. We investigate how our SSL framework can improve feature reuse compared with the original discriminative methods by measuring the similarity of the features before and after fine-tuning on the downstream tasks. If a SSL pretrained model provides features that are similar to the fine-tuned model, it indicates that the SSL approach has learned more useful features. Following (Neyshabur et al., 2020), we employ the Centered Kernel Alignment (CKA) (Kornblith et al., 2019) metric to measure feature similarity. Following (Kaku et al., 2021), we conduct feature reuse evaluations in limited labeled data regimes on classification (10% labeled data of the ChestX-ray14) and segmentation (Montgomery) tasks. We extract features from conv1 and the end of four residual blocks of the ResNet-50 backbone, denoted as layers 1 to 5, and then pass them to a global average pooling layer to compute feature similarity. We fine-tune each SSL model at least five times and report the average CKA score on each downstream task.

Results. Each row in Table 4 presents the per-layer feature similarity between a pretrained model and the corresponding fine-tuned model across two downstream tasks. Our results indicate that DiRA models consistently provide more reusable low/mid-level features (layers 1 to 3) compared with the original discriminative methods. Specifically, DiRA_{MoCo-v2}, DiRA_{Barlow Twins}, and DiRA_{SimSiam} yield an average improvement of 20%, 12%, 14% across the initial three layers in the ChestX-ray14, and 16%, 15%, and 21% in the Montgomery. These results demonstrate DiRA's capability in capturing more fine-grained details, resulting in more reusable features which allows improved generalization to downstream tasks with low-data regimes. Moreover, our analysis reveals that the initial layers (i.e., layers 1 to 3) of DiRA models yield more reusable features than the higher layers (i.e., layers 4 and 5). This observation, in line with previous studies (Asano et al., 2020; Zhao et al., 2021; Neyshabur et al., 2020) and in accordance with our transfer learning results (FIG. 4 and Table 2), highlights the significance of low/mid-level features in transfer learning.

TABLE 5
Comparison with ViT-based fully-supervised baselines:
DiRA outperforms fully-supervised ViT models pretrained
on ImageNet and ChestX-ray14 datasets in six downstream
tasks. All models use the ViT-S/16 backbone. The
best methods are bolded.

| Downstream | Supervised | Supervised | |
|---|---|---|---|
| Task | ChestX-ray14 | ImageNet | DiRA_{MAE} |
| ChestX-ray14 | N/A | 79.88 ± 0.06 | 81.05 ± 0.04 ‡ |
| CheXpert | 85.36 ± 0.21 | 86.82 ± 0.41 | 87.72 ± 0.32 ‡ ‡ |
| SIIM-ACR | 67.57 ± 0.87 | 69.64 ± 0.40 | 70.66 ± 0.54 ‡ ‡ |
| SCR-Heart | 92.51 ± 0.41 | 93.58 ± 0.28 | 94.28 ± 0.15 ‡ ‡ |
| Montgomery | 96.29 ± 0.29 | 97.16 ± 0.07 | 97.58 ± 0.08 ‡ ‡ |
| ChestX-Det | 73.14 ± 0.11 | 74.18 ± 0.12 | 75.16 ± 0.22 ‡ ‡ |

‡ and ‡ denote the statistically significant (p < 0.05) improvement compared with the supervised ImageNet and ChestX-ray14 baselines, respectively. For the supervised ChestX-ray14 model, transfer learning to ChestX-ray14 is not applicable since pretraining and downstream tasks are the same, denoted by “N/A”.

### 4.6. DIRA Elevates Restorative Self-Supervised Learning

Experimental setup. We investigate the effectiveness of our framework in enhancing restorative representation learning in both 2D and 3D settings by applying DiRA to (1) MAE (He et al., 2022) as a representative of masked image modeling (MIM) SSL approaches that leverage a mask-and-reconstruct training mechanism with vision transformers; and (2) TransVW (Haghighi et al., 2021), as a representative of 3D restorative SSL methods for 3D medical imaging, demonstrating superior performance compared with other approaches, including discriminative (Zhuang et al., 2019; Taleb et al., 2020), restorative-only (Zhou et al., 2021b; Chen et al., 2019; Pathak et al., 2016), and restorative and adversarial methods (Tao et al., 2020). To evaluate the generalizability and transferability of the learned representations by our DiRA framework when applied to MAE, we fine-tune both the original MAE and our DiRA-MAE models on six 2D downstream tasks, covering classification (ChestX-ray14 and CheXpert) and segmentation (SIIM-ACR, SCR-Heart, NIH Montgomery, and ChestX-Det). Similarly, for TransVW, we fine-tune both the original TransVW and our DiRA_{TransVW} models on five 3D downstream tasks, including classification (lung nodule and pulmonary embolism false-positive reduction) and segmentation (lung nodule, liver, and brain tumor segmentation).

Results—MAE vs. DiRA_{MAE}: As seen in FIG. 7, DiRA consistently boosts the performance of MAE across downstream tasks. Specifically, DiRA_{MAE} delivers significantly improved performance (p<0.05) compared with the original MAE in ChestX-ray14, CheXpert, ChestX-Det, SIIM-ACR, and SCR-Heart, and comparable performance in NIH Montgomery. FIG. 7 in particular shows comparison with restorative self-supervised learning: we apply DiRA to MAE, a representative of ViT-based masked image modeling self-supervised methods. DiRA empowers MAE to encode more comprehensive representations, leading to significant (p<0.05) performance boosts in five downstream tasks. These results suggest that by incorporating discriminative, restorative, and adversarial learning, DiRA empowers MAE to capture more comprehensive representations, facilitating better generalization across a diverse set of tasks.

Table 5 illustrates the comparison of our DiRA_{MAE} with two fully-supervised baselines—pretrained on ImageNet (Touvron et al., 2021) and ChestX-ray14 datasets using the same backbone (ViT-S/16) as DiRA_{MAE}, across six downstream tasks. Notably, DiRA_{MAE} consistently exhibits significantly better performance (p<0.05) compared with both supervised baselines across all tasks. Specifically, DiRA_{MAE} achieves performance increases of 2.4%, 3.1%, 1.8%, 1.3%, and 2.0% compared with the supervised ChestX-ray14 model in CheXpert, SIIM-ACR, SCR-Heart, Montgomery, and ChestX-Det, respectively. Similarly, DiRA_{MAE} demonstrates performance increases of 1.2%, 0.9%, 1%, 0.7%, 0.4%, and 0.95% compared with the supervised ImageNet model in ChestX-ray14, CheXpert, SIIM-ACR, SCR-Heart, Montgomery, and ChestX-Det. These results suggest that our SSL framework yields more generalizable features compared with supervised pretraining, highlighting its potential for annotation efficiency in pretraining ViT models.

TABLE 6
Comparison with restorative self-supervised learning method: we apply our DiRA
to the TransVW as a representative of 3D restorative-based SSL methods. DiRA
empowers TransVW to capture more fine-grained information, yielding performance boosts
in four 3D downstream tasks. FPR denotes “false positive reduction”. Source:
The DiRA_{TransVW} performance is adopted from (Guo et al., 2022, 2024).

| | Segmentation [IoU (%)] | | | Classification [AUC (%)] | |
|---|---|---|---|---|---|
| Method | Lung nodule | Liver | Brain tumor | Lung nodule FPR | PE FPR |
| Random | 74.05 ± 1.97 | 79.76 ± 5.42 | 59.87 ± 4.04 | 94.25 ± 5.07 | 80.36 ± 3.58 |
| TransVW | 77.33 ± 0.52 | 86.53 ± 1.30 | 68.82 ± 0.38 | 98.46 ± 0.30 | 87.07 ± 2.83 |
| DiRA_{TransVW} | 77.51 ± 1.36 | 86.85 ± 0.81 | 69.57 ± 1.13 | 98.87 ± 0.61 | 86.91 ± 3.27 |
| | (+0.18) | (+0.32) | (+0.75) | (+0.41) | |

Results—TransVW vs. DiRA_{TransVW}: Our results in Table 6 indicate that DiRA framework consistently enhances TransVW's performance across four 3D target task. Specifically, DiRA improves TransVW in lung nodule false positive reduction, lung nodule segmentation, liver segmentation, and brain tumor segmentation, and yields comparable performance in PE false positive reduction task. These results imply that the integration of three learning components in DiRA can enhance self-supervision approaches by capturing more diverse and discriminative visual representations, leading to a greater generalization across different downstream tasks.

To further demonstrate the effectiveness of our DiRA framework in enhancing TransVW, we investigate their learned representations under limited labeled data regimes. To do so, we compared the performance of DiRA_{TransVW} against both TransVW and training from scratch (as the lower bound), using partially labeled data in three 3D downstream tasks: lung nodule segmentation, PE false positive reduction (PE FPR), and lung nodule false positive reduction (lung nodule FPR). For clarity, we identify the minimum required data for training from scratch and TransVW to achieve a performance comparable (based on t-test) to their performance when using the entire training dataset. Moreover, the minimum required data for DiRA_{TransVW} is determined to achieve the performance equivalent to that of training from scratch and TransVW. The results, shown in FIG. 8, indicate that DiRA_{TransVW} exhibits greater label efficiency compared with both training from scratch and TransVW across tasks. In particular, FIG. 8 shows DiRA_{TransVW} exhibits greater label efficiency in 3D downstream tasks, reducing annotation costs by 60%, 57%, and 57% in comparison with training from scratch, and 87%, 16%, and 26% compared with TransVW in lung nodule segmentation, PE FPR, and lung nodule FPR, respectively. The horizontal gray and purple lines demonstrate the performance achieved by training from scratch and TransVW, respectively, when using the entire training data. The gray and purple bars indicate the minimum required data for training from scratch and TransVW to achieve a performance comparable (based on t-test) to their performance when using the entire training dataset. The DiRA_{TransVW} performance is adopted from Guo et al. (2022).

FIG. 8 demonstrates, for instance, in the lung nodule FPR, DiRA_{TransVW} with only 30% of training data yields equivalent performance to training from scratch with 70% of data. Consequently, DiRA_{TransVW} reduces 57% of the annotation cost in the lung nodule FPR task compared with training from scratch. Moreover, DiRA_{TransVW} with 70% of data achieves equal performance with TransVW using 95% of data, resulting in a noteworthy 26% reduction in annotation costs for the lung nodule FPR task compared with TransVW. Overall, DiRA_{TransVW} reduces the annotation cost by 60%, 57%, and 57% in comparison with training from scratch, and 87%, 16%, and 26% compared with TransVW in lung nodule segmentation, PE FPR, and lung nodule FPR, respectively. These results highlight the efficacy of DiRA in capturing more robust and generalizable representations for downstream tasks with a dearth of labeled data.

### 5. ABLATION STUDIES

This section presents the results of our ablation studies. We first investigate the impact of three key components in our SSL framework on transfer learning performance. Subsequently, we conduct complementary feature analysis experiments to gain deeper insights into the effect of fine-grained information on enhancing the discriminative power of features acquired through discriminative self-supervised learning methods. Following that, we conduct experiments to provide deeper insights into the impact of loss coefficients within the overall training objective of DiRA on representation learning. Finally, we examine the impact of pretraining data scale on the transfer performance of contrastive self-supervised learning.

### 5.1. DiRA Realizes a New Paradigm of Collaborative Learning

Experimental setup. Our DiRA framework takes the advantages of three components in representation learning: discriminative, restorative, and adversarial learning. Therefore, we conduct a thorough ablation study to assess the impact of each component on downstream tasks' performances. To do so, for each underlying self-supervised method, i.e. MoCo-v2, Barlow Twins, and SimSiam (referred to as the base), we start with the discriminative learning component and gradually incorporate restorative and the adversarial learning components. Once all three components are unified, they form the completed DiRA models. We pretrain all models on the training set of the ChestX-ray14 dataset and fine-tune them for six downstream tasks, including classification (ChestX-ray14 and CheXpert) and segmentation (SCR-Hear, ChestX-Det, SIIM-ACR, and Montgomery) tasks.

Results. We draw the following observations from the results in Table 7. Firstly, our findings indicate that incorporating a restoration task consistently improves the performance of discriminative self-supervised methods. Specifically, adding [symbol] into training objectives of MoCo-v2, Barlow Twins, and SimSiam boosts the corresponding original methods in all downstream tasks with the exception of SimSiam in ChestXray14, which shows slight performance degradation. It should be noted that the performance gap observed in SimSiam for the ChestXray14 task is later compensated after adding [symbol] , which highlights the collaborative learning between the restorative and adversary components within our framework. Secondly, our results demonstrate that incorporating an adversarial discriminator into the restoration component offers significant benefits, improving the performance of all methods across six different downstream tasks. Our observations suggest that unifying three SSL learning components in DiRA models significantly improves the original self-supervised methods through preserving more fine-grained contextual information from medical images.

TABLE 7
Ablation study on different components of DiRA: we study the impact of each learning component of DiRA, including discrimination, restoration,
and adversary, in four downstream tasks. Incremental addition of restorative learning ( [symbol] ) to discriminative learning leads to consistent
performance improvements. Furthermore, equipping models with adversarial learning ( [symbol] ) yields further performance boosts across all tasks.

| | Pretraining | | | | Classification | | Segmentation | | | |
|---|---|---|---|---|---|---|---|---|---|---|
| Base | dataset | [symbol] | [symbol] | [symbol] | ChestX-ray14 | CheXpert | SIIM-ACR | Montgomery | SCR-Heart | ChesX-Det |
| MoCo-v2 | ChestX- | ✓ | x | x | 80.36 ± 0.26 | 86.42 ± 0.42 | 67.89 ± 1.14 | 98.03 ± 0.22 | 92.45 ± 0.92 | 70.83 ± 1.30 |
| | ray14 | ✓ | ✓ | x | 80.72 ± 0.29 ↑ | 86.86 ± 0.37 ↑ | 68.16 ± 1.07 ↑ | 98.19 ± 0.08 ↑ | 93.88 ± 0.58 ↑ | 70.99 ± 1.27 ↑ |
| | | ✓ | ✓ | ✓ | 81.12 ± 0.17 ↑ | 87.59 ± 0.28 ↑ | 69.24 ± 0.41 ↑ | 98.24 ± 0.09 ↑ | 94.17 ± 1.01 ↑ | 72.84 ± 1.13 ↑ |
| Barlow | ChestX- | ✓ | x | x | 80.45 ± 0.29 | 86.90 ± 0.62 | 69.71 ± 0.34 | 98.13 ± 0.13 | 91.87 ± 1.88 | 71.80 ± 1.18 |
| Twins | ray14 | ✓ | ✓ | x | 80.86 ± 0.16 ↑ | 87.44 ± 0.33 ↑ | 69.83 ± 0.29 ↑ | 98.15 ± 0.14 ↑ | 92.15 ± 1.35 ↑ | 73.21 ± 1.24 ↑ |
| | | ✓ | ✓ | ✓ | 80.88 ± 0.30 ↑ | 87.50 ± 0.27 ↑ | 69.87 ± 0.68 ↑ | 98.16 ± 0.06 ↑ | 93.19 ± 0.93 ↑ | 73.49 ± 0.78 ↑ |
| SimSiam | ChestX- | ✓ | x | x | 79.62 ± 0.34 | 83.82 ± 0.94 | 67.58 ± 1.89 | 97.72 ± 0.27 | 92.26 ± 1.72 | 70.49 ± 1.07 |
| | ray14 | ✓ | ✓ | x | 79.41 ± 0.42 ↓ | 84.45 ± 0.46 ↑ | 68.35 ± 1.16 ↑ | 98.02 ± 0.21 ↑ | 93.56 ± 0.61 ↑ | 70.60 ± 1.64 ↑ |
| | | ✓ | ✓ | ✓ | 80.44 ± 0.29 ↑ | 86.04 ± 0.43 ↑ | 68.76 ± 0.69 ↑ | 98.17 ± 0.11 ↑ | 93.75 ± 0.98 ↑ | 72.10 ± 1.36 ↑ |

### 5.2. Fine-Grained Self-Supervised Learning Yields More Discriminative Features

Instance discrimination methods treat each image as a distinct class and aim to learn an optimal embedding space where all instances are well-separated. Effective separation in the embedding space indicates that the SSL method has learned more discriminative features, leading to better generalization to various downstream tasks. But, given the fundamental anatomical similarity of medical images, the global representations captured by standard instance discrimination methods may be insufficient to distinguish similar appearing medical images from one another. In fact, such coarse-grained representations may lead to a sub-optimal embedding space, which does not generalize well to different downstream tasks. We believe that exploiting the diversity in the local context of images can empower instance discrimination learning with more discriminative features, distinguishing individual images more effectively.

Experimental setup. To demonstrate the impact of fine-grained contextual information for enhancing instance discrimination, we integrate restorative learning with three instance discrimination methods, namely MoCo-v2, Barlow Twins, and SimSiam, and compare the distribution of distances between features learned by them with the original instance discrimination counterparts. To do so, we first utilize the pretrained models to extract features from images in the testing set of ChestX-ray14 dataset. We extract features from the last layer of the ResNet-50 backbone and apply global average pooling to obtain a feature vector for each image. Then, we normalize the features and compute all pairwise distances between features of individual images using the Euclidean distance metric. Finally, we visualize the distance distributions with Gaussian kernel density estimation (KDE). A SSL approach that captures more diverse and discriminative representations, yields an embedding space with greater feature distances.

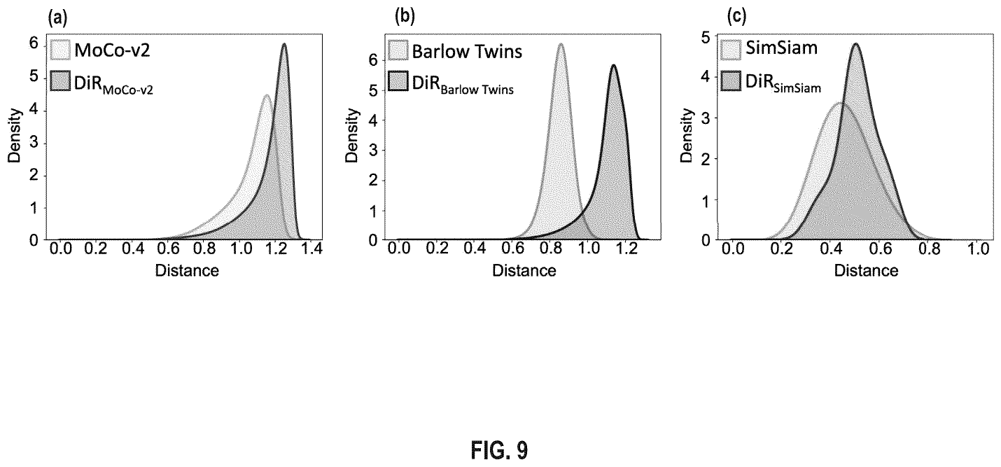

Results. As shown in FIG. 9, exploiting the diversity in the local context of images empowers the instance discrimination methods to provide substantially increased feature distances in comparison with the original methods. In particular, the mean distance of MoCo-v2, Barlow Twins, and SimSiam increased by 9%, 30%, and 11%, respectively. The enhanced separation of images in the embedding space implies that fine-grained SSL provides more discriminative features by adequately capturing finer contextual information from individual images.

### 5.3. Impact of Loss Coefficients

Experimental setup. We perform a comprehensive ablation study to explore various scales for the discriminative, restorative, and adversarial loss coefficients (i.e. λ_{dis}, λᵣₑₛ, and λ_{adv}) within the overall training objective of DiRA, aiming to provide deeper insights into the impact of these hyperparameters on representation learning. To do so, we train our DiRA_{MoCo-v2} model with different scales for each loss coefficient and monitor each model's performance across various downstream tasks, including SIIM-ACR, SCR-Heart, ChestX-Det, and 10% of ChestX-ray14 datasets. In particular, we start by examining different scales (i.e. 0.1, 1, and 10) for λ_{dis}. After selecting the optimal λ_{dis} as per our ablation study, we evaluate different scales (i.e. 1, 10, and 100) for the λᵣₑₛ. Finally, with λ_{dis} and λᵣₑₛ set to their optimal values, we explore different scales (i.e. 0.0001, 0.001, and 0.01) for λ_{adv}. For a comprehensive analysis, we also include the results of the original MoCo-v2 as the performance lower-bound for each downstream task.

### Results

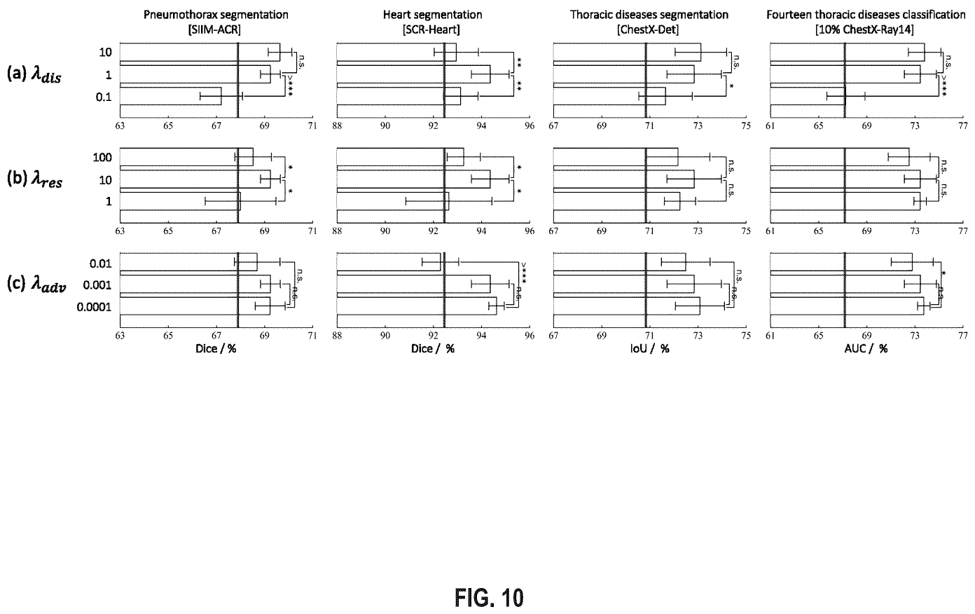

FIG. 10 shows an ablation study on loss coefficient: we examine various scales for the (a) discriminative loss coefficient (λ_{dis}), (b) restorative loss coefficient (λᵣₑₛ), and (c) adversarial loss coefficients (adv) using our DiRA_{MoCo-v2} model. Using different values of λ_{dis}, λᵣₑₛ, and λ_{adv}, DiRA_{MoCo-v2} models demonstrate superior performance compared with the original MoCo-v2 model (as indicated by the blue vertical lines) in nearly all cases, highlighting the robustness of our DiRA framework to these hyperparameters. Nevertheless, a judicious tuning of these coefficients could facilitate further performance improvement.

The following observations can be drawn from the results in FIG. 10. (1) As seen in FIGS. 10-a, 10-b, and 10-c, using different values of λ_{dis}, λᵣₑₛ, and λ_{adv}, DiRA_{MoCo-v2} models consistently outperform the original MoCo-v2 model in almost all cases. This finding underscores the robustness of the subject SSL framework to these hyperparameters, indicating that the integration of the three components consistently delivers top performance. (2) As seen in FIG. 10-a, reducing λ_{dis} to 0.1 can markedly decrease the downstream performance, particularly in the classification task (i.e. ChestX-ray14 dataset), compared with adhering to a coefficient value of 1. Conversely, increasing λ_{dis} from 1 to 10 does not consistently translate to improved performance in downstream tasks. In fact, the model with a higher discrimination loss coefficient exhibits statistically comparable or inferior performance to the model with the unscaled discrimination loss across various downstream tasks. These results suggest that setting λ_{dis} to 1 is a favorable choice for this hyperparameter. (3) As seen in FIG. 10-b, decreasing λᵣₑₛ from 10 to 1 leads to a decrease in downstream performance, especially for segmentation tasks in SIIM-ACR and SCR-Heart datasets, compared with using a larger coefficient value of 10.

TABLE 8
We assess the impact of pretraining data scale on the performance
of contrastive SSL in medical imaging by evaluating MoCo-v2
models pretrained on varying number of unlabeled images
collected from different data sources.

| #Images | Source |
|---|---|
| 86K | ChestX-ray14 |
| 246K | ChestX-ray14, PadChest |
| 469K | ChestX-ray14, PadChest, CheXpert |
| 837K | ChestX-ray14, PadChest, CheXpert, MIMIC-CXR |
| 4.8M | Hospital-scale dataset (Cho et al., 2023) |

This trend may be attributed to the potential reduction in the model's restoration learning capacity relative to its discrimination learning capacity when using a lower value for λᵣₑₛ. Nevertheless, increasing λᵣₑₛ from 10 to 100 reduces performance across tasks, with a more pronounced impact on the classification performance in ChestX-ray14 dataset. These results imply that an excessive submissiveness or dominance of the restoration loss over the discrimination loss may not be advantageous. Instead, maintaining a balance between them appears more suitable for different downstream tasks. (4) As seen in FIG. 10-c, decreasing λ_{adv} from 0.001 to 0.0001 enhances the average performance across downstream tasks, with no significant difference based on our statistical analysis. Conversely, we observed that increasing the value of λ_{adv} from 0.001 to 0.01 decreases downstream performance across all tasks, with a statistically significant decline in SCR-Heart and ChestX-ray14 datasets. These observations, in line with previous studies (Pathak et al., 2016; Tao et al., 2020), underscore the preference for lower adversarial loss coefficient values over the restoration loss coefficient in generative-adversarial learning settings. In summary, our ablation experiments imply that our DiRA framework exhibits robustness to variations in loss coefficient hyperparameters; nonetheless, a judicious selection of these coefficients has the potential to further optimize performance.

### 5.4. Impact of Pretraining Data Scale on Contrastive Learning

The capability to harness large-scale data without relying on human annotations is a pivotal advantage of self-supervised learning. The efficacy of pretraining self-supervised models with massive photographic images, particularly using contrastive approaches, has been proved in various vision tasks (He et al., 2020; Goyal et al., 2021). However, given the marked differences between photographic and medical images, it remains unexplored to what extent contrastive SSL techniques benefit from large-scale pretraining datasets when applied to medical tasks. In contrast to photographic images, medical images (e.g. chest radiographs) display great similarities in global anatomy. We hypothesize that contrasting medical images against each other, even with a larger dataset, may not enable the model to capture more diverse representations. Consequently, contrastive learning may not fully realize the potential benefits of large-scale pretraining data in medical imaging. To test this hypothesis, we systematically study the scaling behaviors of contrastive learning for medical imaging through extensive experiments with pretraining data ranging from modest to large-scales.

Experimental setup: We investigate the impact of pretraining data size on the transfer performance of contrastive self-supervised learning in medical imaging. To do so, we evaluate the transferability of MoCo-v2 models pretrained with varying amounts of unlabeled data, ranging from modest datasets (i.e., 86K samples) to considerably larger ones (i.e., 4.8M samples), across a diverse set of downstream tasks. Specifically, we assemble data from four public chest radiographic datasets—ChestX-ray14, PadChest (Bustos et al., 2020), CheXpert, and MIMIC-CXR (Johnson et al., 2019)—to gradually form larger-scale datasets for pretraining. As summarized in Table 8, we employ these datasets to pretrain MoCo-v2 with 86K, 246K, 469K, and 837K samples. Moreover, we examine the MoCo-v2 model pretrained on a collection of 4.8M images provided by Cho et al. (2023). All five pretrained models under the study employ the same backbone (i.e. ResNet-50), and are fine-tuned for five downstream tasks, encompassing classification tasks on ChestX-ray14 and CheXpert datasets, as well as segmentation tasks on SCR-Heart, ChestX-Det, and SIIM-ACR datasets.

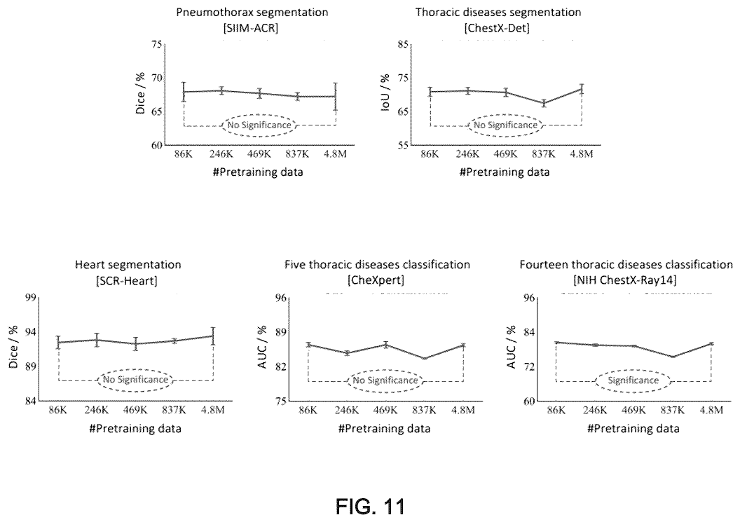

Results. Our results in FIG. 11 indicate that increasing the amount of pretraining data does not provide additional benefits to downstream performance. Specifically, for segmentation tasks, we observe that increasing the pretraining data from 86K to 4.8M does not result in statistically significant changes in performance. Notably, the model pretrained with 86K images yields comparable performance to the one pretrained with 4.8M images in SIIM-ACR, SCR-Heart, and ChestX-det applications. Similarly, for classification tasks, increasing the amount of pretraining data from 86K to 4.8M either fails to increase or even leads to slight decrease in performance. For instance, when comparing models pretrained with 86K and 4.8M images, the former demonstrates superior performance in ChestX-ray14 and equivalent performance in CheXpert. We surmise that contrastive methods might face challenges in effectively distinguishing medical images, which consist of similar global structures, from one another. Furthermore, scaling the number of images, which could introduce variations in data distribution, might exacerbate this challenge, potentially leading to a negative impact on the generalizability of features in models pretrained with larger datasets. In summary, our results suggest that scaling pretraining data standalone for contrastive learning may not translate to additional boosts in transfer performance; thereby, exploring more advanced techniques for optimizing contrastive learning, as well as designing pretext tasks that take into account the unique properties of medical images, holds the potential to fully unlock the benefits of self-supervised pretraining at scale for medical imaging.

### 6. RELATED WORKS

### 6.1. Discriminative Self-Supervised Learning

Discriminative methods aim to train models that can distinguish samples of different (pseudo) classes. Discriminative methods can be divided into class-level and instance-level discrimination. Class-level discrimination methods generate target class labels based on relative positions of patch images (Doersch et al., 2015; Noroozi and Favaro, 2016), orientations of objects within images (Gidaris et al., 2018), or cluster assignments (Zhan et al., 2020; Caron et al., 2018, 2021a), and then train a model to predict the pseudo labels of the input images. On the other hand, instance-level discrimination has emerged as a prominent approach for learning more robust representations. The pivotal idea is to consider each image as a unique class and train a model to align the representations of the augmented views from the same image, enforcing invariance to image transformations. Instance-level discrimination methods exploit different strategies to prevent collapsing solutions. Contrastive methods (He et al., 2020; Chen et al., 2020b; Wu et al., 2018; Chen et al., 2020a; Chuang et al., 2022; Zhang et al., 2022a; Caron et al., 2021a; Ye et al., 2019; Li et al., 2021) employ samples from different images or different clusters as negative pairs to distinguish dissimilar samples from each other. Self-distillation methods (Chen and He, 2021; Grill et al., 2020; Caron et al., 2021b; Gidaris et al., 2021) train a student network to predict the representations of a teacher network, and the teacher network's weights are not updated by back-propagation but instead are a moving average of the student network's weights or shared with the student network. Information maximization methods (Zbontar et al., 2021; Ermolov et al., 2021; Bardes et al., 2022; Zhang et al., 2022b) maximize the information content of the representations while also preserving the similarity between different views of the same image in the embedding space. Additionally, recent works (Xie et al., 2021b; Caron et al., 2021b; Chen et al., 2021c) have explored instance discrimination for pre-training vision transformers. However, both class-level and instance-level discrimination approaches yield global image representations, which may limit their ability to generalize to the tasks that require finer-grained representations (Wang et al., 2021; Xie et al., 2021c,a). By contrast, our DiRA incorporates restorative and adversarial learning, which not only enhances discriminative learning but also captures fine-grained representations required for medical imaging tasks.

### 6.2. Restorative and Adversarial Self-Supervised Learning

Restorative methods seek to preserve the instance-level fidelity, ensuring the faithful reconstruction of individual data samples. These methods employ element-wise metrics to measure image similarity at pixel-level. A broad variety of restorative methods apply degradation, such as noise (Vincent et al., 2008), decolorization (Larsson et al., 2017), or masking (Pathak et al., 2016), to perturb or remove some parts of the images, and then train a model to reconstruct the original images or the missing parts. Additionally, Generative Adversarial Networks (GANs) (Goodfellow et al., 2014), initially designed for image generation, have shown promise in acquiring transferable knowledge (Chen et al., 2020c; Donahue and Simonyan, 2019; Dumoulin et al., 2017). Adversarial learning methods aim to preserve set-level fidelity, ensuring the reconstruction is faithful to the entire training dataset (Parmar et al., 2021). Indeed, adversarial learning measures the image similarity using higher-level and sufficiently invariant metrics compared with pixel-wise metrics used by restorative learning (Larsen et al., 2016). This is accomplished by learning a function capable of contrasting (classifying) the set of training images with the set of generated images, thereby encouraging the reconstructed images to align with the true data distribution. With the advent of large-scale vision transformers (Dosovitskiy et al., 2021), masked image modeling (MIM) has emerged as a promising self-supervised technique. MIM methods (Xie et al., 2022; He et al., 2022) are built upon the mask-and-reconstruct pretext task, drawing inspiration from the BERT (Devlin et al., 2019) in Natural Language Processing. Despite achieving impressive results, restorative methods primarily capture local spatial correlations within individual image samples, disregarding the relationships among different samples, which consequently leads to less discriminative representations (Zhou et al., 2022; Mishra et al., 2022). Our DiRA addresses this limitation by incorporating discriminative learning, which complements restorative and adversarial learning through explicitly modeling the relationship between all samples.

### 6.3. Self-Supervised Learning in Medical Image Analysis

Self-supervised learning has gained considerable attention in medical vision, primarily due to its potential in mitigating the challenges posed by the limited availability of labeled data. Building upon the success of instance discrimination SSL in vision tasks, recent studies have delved into employing instance discrimination for medical tasks, including adjusting MoCo-based contrastive learning for chest X-ray classification (Zhou et al., 2020; Sowrirajan et al., 2021; Hosseinzadeh Taher et al., 2023; Cho et al., 2023), expanding SimCLR by using multiple scans of the same patients as positive pairs (Azizi et al., 2021), integrating local and global contrastive learning (Chaitanya et al., 2020), and incorporating intermediate features in contrastive learning (Kaku et al., 2021) Another line of research centers around restorative-based approaches, which can be divided into three primary categories: restorative-only methods (Zhou et al., 2021b; Chen et al., 2019; Chang et al., 2022; Xiao et al., 2023), restorative and adversarial methods (Tao et al., 2020), and discriminative and restorative methods (Hosseinzadeh Taher et al., 2022; Haghighi et al., 2021; Zhou et al., 2021a; Tang et al., 2022). Particularly, discriminative and restorative methods, such as TransVW (Haghighi et al., 2021), have demonstrated superiority over restorative-only and restorative and adversarial methods. DiRA distinguishes itself from previous works by (1) uniting discriminative, restorative, and adversarial learning simultaneously in a unified framework; and (2) offering a versatile representation learning framework that seamlessly aligns with existing discriminative, restorative, and adversarial methods, regardless of their objective functions.

### 6.4. Our Previous Work

Haghighi et al. (2022) first introduced the integration of discriminative, restorative, and adversarial learning into a unified framework, pioneering a new paradigm of collaborative learning for SSL. The subject inventive concept substantially expands upon the preliminary version and incorporates the following notable enhancements:
1. We have extended our transfer learning evaluations to 12 medical tasks, showcasing the superior generalizability of DiRA models compared with existing state-of-the-art SSL approaches across distribution shifts, diseases, organs, and modalities (see Section 4.1).
2. We have conducted extensive experiments in more restricted data regimes and added disease segmentation task (ChestX-Det), demonstrating the label efficiency of DiRA models for medical image classification and segmentation tasks (see Section 4.2).
3. We have provided quantitative evaluations for weakly-supervised disease localization, indicating the effectiveness of DiRA models in encoding essential cues for disease localization compared with existing SSL methods (see Section 4.3).
4. We have expanded the comparison of DiRA models with fully-supervised baselines by adding two new downstream tasks, namely heart and thoracic diseases segmentation, highlighting the superiority of DiRA models over their fully-supervised counterparts (see Section 4.4).
5. We have analyzed feature quality in terms of feature reuse, revealing that DiRA models capture highly reusable low/mid-level features for medical downstream tasks (see Section 4.5).
6. We have conducted comprehensive ablation studies on the impact of each learning components in DiRA, demonstrating the synergy of three learning components for collaborative learning (see Section 5.1).
7. We have investigated the impact of fine-grained contextual details on instance discrimination pretext tasks, demonstrating that the diversity of local information empowers instance discrimination methods to capture more discriminative features (see Section 5.2)
8. We have adopted MAE, as a representative of ViT-based masked autoencoders, into our DiRA framework, demonstrating the efficiency of DiRA in enhancing MAE across diverse classification and segmentation downstream tasks (see Section 4.6).
9. We have compared our DiRA_{MAE} model with ViT backbone against two fully-supervised baseline counterparts—pretrained on ImageNet and ChestX-ray14 datasets with the same ViT backbone—showcasing superior performance of DiRA_{MAE} over supervised baselines in six downstream tasks (see Section 4.6).
10. We have investigated the efficacy of our DiRA_{TransVW} under limited labeled data regimes in 3D downstream tasks, revealing that DiRA_{TransVW} achieves greater label efficiency through a significant reduction in annotation costs compared with TransVW (see Section 4.6).
11. We have conducted a comprehensive ablation study on the discriminative, restorative, and adversarial loss coefficients, providing deeper insights into the impact of these hyperparameters on the learning process (see Section 5.3).
12. We have systematically investigated the impact of pretraining data scale on the transfer performance of contrastive self-supervised learning across various medical imaging tasks, revealing that increasing the amount of pretraining data may not yield additional benefits to downstream performance (see Section 5.4).

### 7. NON-LIMITING CONCLUSIONS

This paper introduces DiRA, the first self-supervised framework that unites discriminative, restorative, and adversarial learning in a unified manner. The core contribution of DiRA stems from the insights we have gained into the synergistic effects of these three SSL approaches in collaborative learning. Our extensive experiments on a diverse suite of medical tasks demonstrate that DiRA encourages collaborative learning among three learning ingredients, resulting in more generalizable representation for different medical tasks, superior performance over supervised and self-supervised baselines, and higher reusability of low/mid-level features. Given DiRA's generalizability, we envision it will take a fundamental step towards developing universal representations for medical imaging. Our DiRA offers remarkable performance gains; nevertheless, in our experiments, we kept the restorative learning task fixed while exploring various formulations of discriminative learning. Future research into a broader range of restoration tasks and optimal collaborative learning strategies holds the potential to yield even stronger representations for medical imaging applications.

### APPENDIX A. ADDITIONAL QUALITATIVE RESULTS

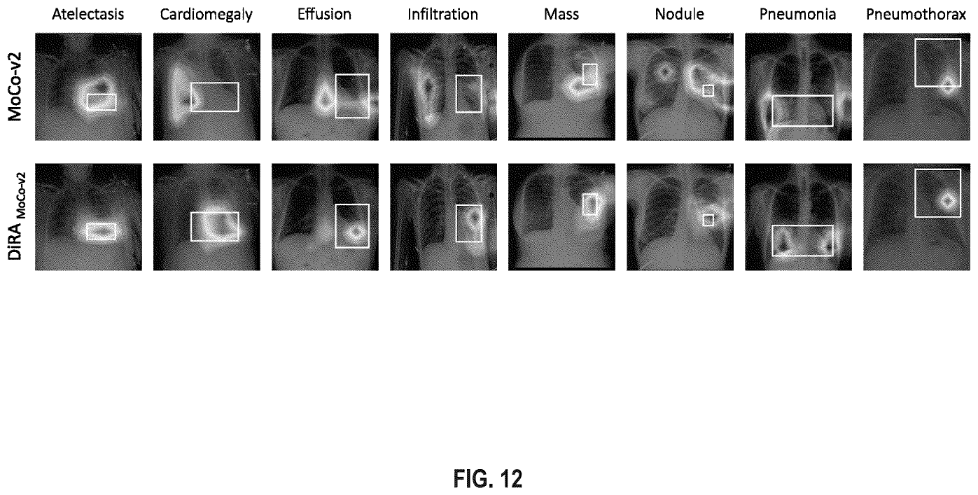

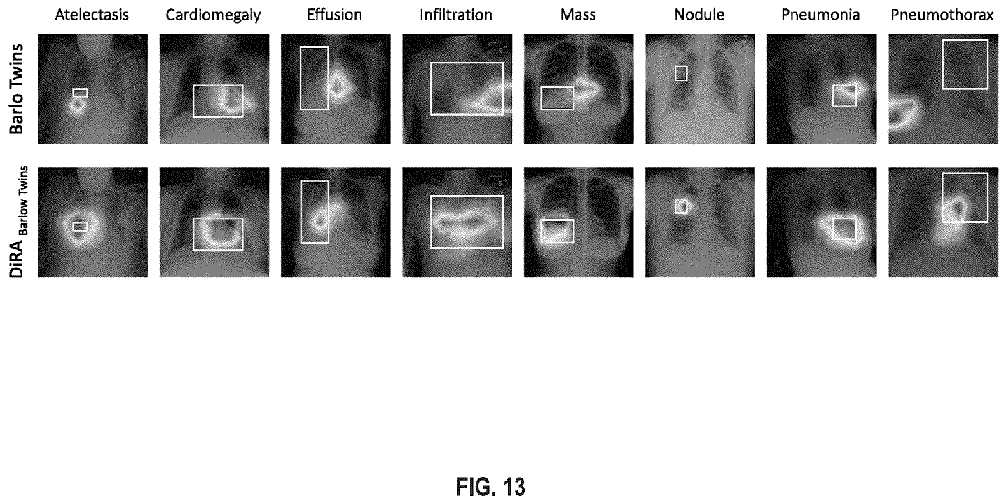

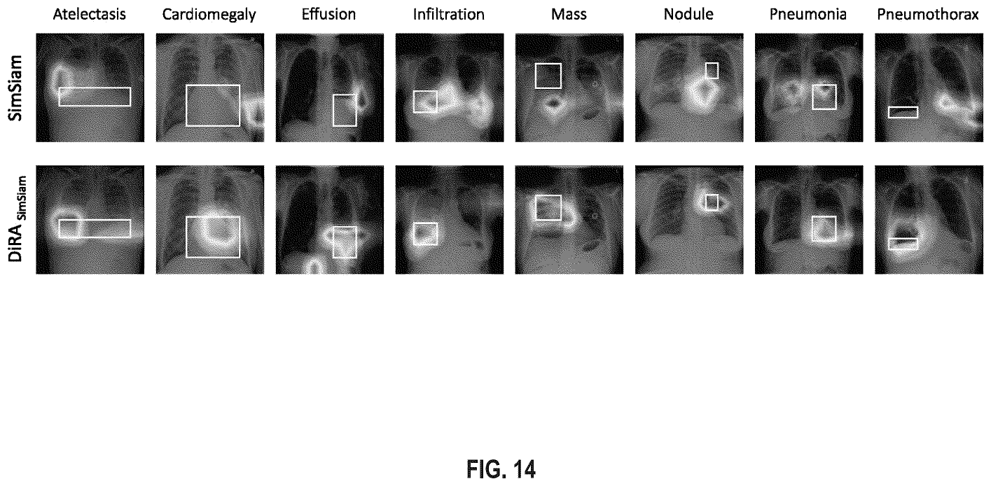

FIGS. 12-14 present the visualizations of heatmaps generated by DiRA and the corresponding discriminative models for 8 thoracic pathologies in the ChestX-ray14 dataset. As seen, DiRA models provide more accurate pathology localization compared with the underlying discriminative methods. These results demonstrate the impact of restorative learning in providing fine-grained features that are useful for disease localization.

### APPENDIX B. IMPLEMENTATION

### B.1. Pretraining Settings

We apply DiRA to five existing self-supervised methods (Chen et al., 2020a; Chen and He, 2021; Zbontar et al., 2021; He et al., 2022; Haghighi et al., 2021). To be self-contained, we will explain each method briefly here. Also, we provide additional pretraining details that supplements Section 3.1.

MoCo-v2 (Chen et al., 2020a): We adopt MoCo-v2-a representative of contrastive learning methods, into our framework. MoCo leverages a momentum encoder to ensure the consistency of negative samples as they evolve during training. Moreover, a queue K={k₁, k₂, . . . , k_{N}} is utilized to store the representations of negative samples. The discrimination task is to contrast representations of positive and negative samples. As MoCo-v2 is adopted in DiRA, the encoder f_{θ} and projection head h_{θ} are updated by back-propagation, while f_{ξ} and h_{θ} are updated by using an exponential moving average (EMA) of the parameters in f_{θ} and h_{θ}, respectively. The discrimination branch is trained using InfoNCE loss (van den Oord et al., 2019), which for a pair of positive samples x₁ and x₂ defined as follows:

$${ℒ}_{dis} = - log \frac{exp ( {z}_{1} \cdot {z}_{2} / τ )}{{\sum }_{n = 0}^{N} exp ( {z}_{1} \cdot {k}_{n} / τ )} \qquad ( B \text{.1} )$$

where z₁=h_{θ}(f_{θ}(x₁)) and z₂=h_{ξ}(f_{ξ}(x₂)), τ is a temperature hyperparameter, and N is the queue size. Following (Chen et al., 2020a), f_{θ} is a standard ResNet-50 and h_{θ} is a two-layer MLP head (hidden layer 2048-d, with ReLU). Moreover, when adopting MoCo-v2 in DiRA, f_{θ}, h_{θ}, and g_{θ} are optimized using SGD with an initial learning rate of 0.03, weight decay 0.0001, and the SGD momentum 0.9.

SimSiam (Chen and He, 2021): We adopt SimSiam—a representative of self-distillation methods, into our framework. SimSiam trains the model without negative pairs and directly maximizes the similarity of two views from an image using a simple siamese network followed by a predictor head. To prevent collapsing solutions, a stop-gradient operation is utilized. As such, the model parameters are only updated using one distorted version of the input, while the representations from another distorted version are used as a fixed target. As SimSiam is adopted in DiRA, the encoder f_{θ} and projection head h_{θ} share weights with f_{ξ} and h_{ξ}, respectively. The model is trained to maximize the agreement between the representations of positive samples using negative cosine similarity, defined as follows:

$$𝒟 ( {z}_{1} , {y}_{2} ) = - \frac{{z}_{1}}{{‖ {z}_{1} ‖}_{2}} \cdot \frac{{y}_{2}}{{‖ {y}_{2} ‖}_{2}} \qquad ( B \text{.2} )$$

where z₁=h_{θ}(f_{θ}(x₁)) and z₁=h_{ξ}(f_{ξ}(x₂)). The discrimination branch is trained using a symmetrized loss as follows:

$${ℒ}_{dis} = \frac{1}{2} 𝒟 ( {z}_{1} , stopgrad ( {y}_{2} ) ) + \frac{1}{2} 𝒟 ( {z}_{2} , stopgrad ( {y}_{1} ) ) \qquad ( B \text{.3} )$$

where stopgrad means that y₂ is treated as a constant in this term. Following (Chen and He, 2021), f_{θ} is a standard ResNet-50 and h_{θ} is a three-layer projection MLP head (hidden layer 2048-d), followed by a two-layer predictor MLP head. Moreover, when adopting SimSiam in DiRA, f_{θ}, h_{θ}, and g_{θ} are optimized using SGD with a linear scaling learning rate (Ir×BatchSize/256). The initial learning rate is 0.05, weight decay is 0.0001, and the SGD momentum is 0.9.

Barlow Twins (Zbontar et al., 2021): We adopt Barlow Twins-a representative of information maximization methods, into our framework. Barlow Twins makes the cross-correlation matrix computed from two siamese branches close to the identity matrix. By equating the diagonal elements of the cross-correlation matrix to 1, the representation will be invariant to the distortions applied to the samples. By equating the off-diagonal elements of the cross-correlation matrix to 0, the different components of the representation will be decorrelated, so that the output units contain non-redundant information about the sample. The discrimination loss is defined as follows:

$${ℒ}_{dis} = {\sum }_{i} {( 1 - {𝒞}_{ii} )}^{2} + λ {\sum }_{i} {\sum }_{i \neq j} {𝒞}_{ij}^{2} \qquad ( B \text{.4} )$$

where C is the cross-correlation matrix computed between the outputs of the h_{θ} and h_{ξ} networks along the batch dimension. λ is a coefficient that determines the importance of the invariance and redundancy reduction terms in the loss. Following (Zbontar et al., 2021), f_{θ} is a ResNet-50 and h_{θ} is a three-layer MLP head. Moreover, when adopting Barlow Twins in DiRA, f_{θ}, h_{θ}, and g_{θ} are optimized using LARS optimizer with the learning rate schedule similar to (Zbontar et al., 2021).

MAE (He et al., 2022): We adopt MAE—a representative of ViT-based masked image modeling methods, into our framework. MAE masks a substantial portion of random patches from input images and then reconstructs the missing patches at the pixel level. It utilizes an encoder-decoder architecture, where the encoder follows the ViT (Dosovitskiy et al., 2021) design, and the decoder is a lightweight vision transformer. The encoder-decoder adopts an asymmetric design: the encoder processes only the visible patches, while the decoder reconstructs the original images from the encoded visible patches and mask tokens. Our training settings for MAE closely follow (Xiao et al., 2023). The image is divided into non-overlapping grid patches, and these patches are randomly masked, with a masking ratio of 90%. Positional embeddings are added to both visible and masked patches to retain information about their original locations in the image. The encoder is then applied to the visible patches, mapping them to latent representations. Subsequently, the decoder processes both visible and masked image patches to restore the original images. For [symbol] , we compute Mean squared error (MSE) between the reconstructed and original images in the pixel space, averaged over masked patches. As we adopt MAE into DiRA, f_{θ} is a standard ViT-small with patch size 16×16 and g_{θ} is a vision transformer with depth 2 and embedding dimension 512. Following (Chen et al., 2021c), h_{θ} is a three-layer MLP head (hidden layer 4096-d with ReLU and Batch normalization, output dimension 128). f_{θ} and f_{ξ} share weights; similarly, h_{θ} and h_{ξ} share weights. For [symbol] _{dis}, we use InfoNCE as detailed in Chen et al. (2021c). During the pretraining, f_{θ}, h_{θ}, and g_{θ} are optimized using AdamW optimizer with β₁=0.9, β₂=0.95, weight decay 0.05, and base learning rate 1.5e-4. Learning rate is warmed up for 20 epochs, and then decayed following the cosine annealing strategy.

TransVW (Haghighi et al., 2021): TransVW defines the similar anatomical patterns within medical images as anatomical visual words, and combines the discrimination and restoration of visual words in a single loss objective. As TransVW is adopted in DiRA, the encoder f_{θ} and projection head h_{θ} are identical to f_{ξ} and h_{ξ}, respectively. In particular, the discrimination branch is trained to classify instances of visual words according to their pseudo class labels using the standard cross-entropy loss:

$${ℒ}_{dis} = - \frac{1}{B} {\sum }_{b = 1}^{B} {\sum }_{c = 1}^{C} {𝒴}_{bc} log {𝒫}_{bc} \qquad ( B \text{.5} )$$

where B denotes the batch size; C denotes the number of visual words classes; [symbol] and [symbol] represent the ground truth (one-hot pseudo label vector obtained from visual word classes) and the prediction of h_{θ}, respectively. Following (Haghighi et al., 2021), we use 3D U-Net as the f_{θ} and g_{θ}. h_{θ} includes a set of fully-connected layers followed by a classification head. f_{θ} and g_{θ} are trained with the same setting as (Haghighi et al., 2021).

Joint training process: Following (Chaitanya et al., 2020; Chen et al., 2021b), we perform the overall pretraining with the discrimination, restoration, and adversarial losses in a gradual evolutionary manner. First, the encoder f_{θ} along with projector h_{θ} are optimized using the discrimination loss [symbol] according to the learning schedule of the original discriminative methods (Chen et al., 2020a; Chen and He, 2021; Zbontar et al., 2021; Haghighi et al., 2021), empowering the model with an initial discrimination ability. Then, the restoration and adversarial losses are further fused into the training process incrementally. To stabilize the adversarial training process and reduce the noise from imperfect restoration at initial epochs (Chen et al., 2021b), we first warm up the f_{θ} and g_{θ} using the [symbol] + [symbol] , and then add the adversarial loss [symbol] to jointly train the whole framework; the optimization of the framework by incorporation of [symbol] and [symbol] takes up to 800 epochs. Following (Zhou et al., 2021a), we use the early-stop technique on the validation set, and the checkpoints with the lowest validation loss are used for fine-tuning.

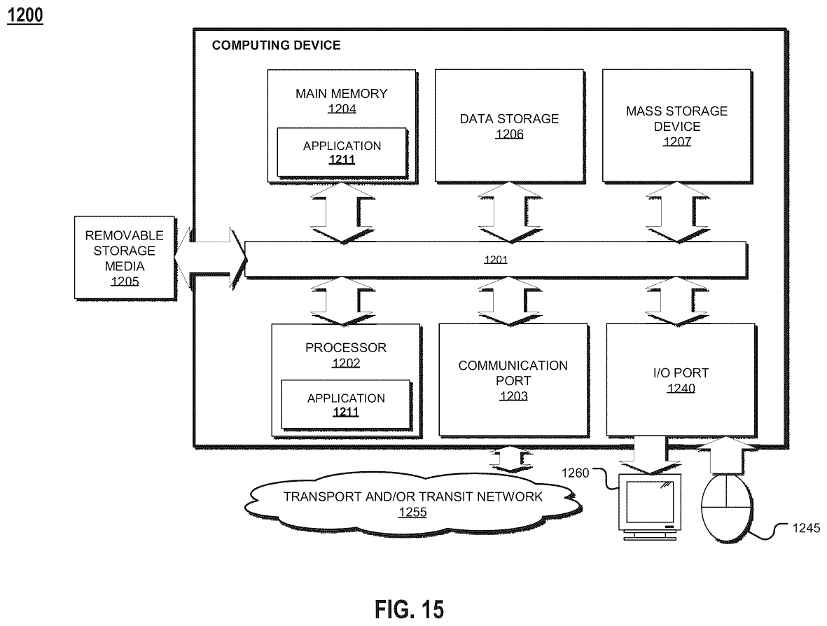

Exemplary Computing Device: Referring to FIG. 15, a computing device 1200 is illustrated which may can be configured, via one or more of an application 1211 or computer-executable instructions, to execute functionality described herein. More particularly, in some embodiments, aspects of the methods herein may be translated to software or machine-level code, which may be installed to and/or executed by the computing device 1200 such that the computing device 1200 is configured to execute functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201 but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN) but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

The described methods, processes, operations, and associated actions may also be performed in various orders in addition to the order described in this application, in parallel, and/or simultaneously. The described systems are exemplary in nature and may include additional elements and/or omit elements. Furthermore, references to or “one example” of the present disclosure are not intended to be interpreted as excluding the existence of additional embodiments that also incorporate the recited features. It will be understood that when a certain part or process “includes” a certain component or operation, that part or process does not exclude another component or operation. While illustrative examples of the name screening techniques using phonetic embeddings have been described herein including systems, devices, and the like, it is to be understood that various other adaptations and modifications may be made within the spirit and the scope of the examples herein. Additionally, it is appreciated that while specific graphics are shown and described, such graphics are illustrative and exemplary and are not intended to limit the scope of this disclosure.

The foregoing description has been directed to specific examples. It will be apparent, however, that other variations and modifications may be made to the described examples, with the attainment of some or all of their advantages. For instance, it is expressly contemplated that the components and/or elements described herein can be implemented as software being stored on a tangible (non-transitory) computer-readable medium, devices, and memories (e.g., disks/CDs/RAM/EEPROM/etc.) having program instructions executing on a computer, hardware, firmware, or a combination thereof. Further, methods describing the various functions and techniques described herein can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on. In addition, devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example. Instructions, media for conveying such instructions, computing resources for executing them, and other structures for supporting such computing resources are means for providing the functions described in these disclosures. Accordingly, this description is to be taken only by way of example and not to otherwise limit the scope of the examples herein. Therefore, it is the object of the appended claims to cover all such variations and modifications as come within the true spirit and scope of the examples herein.

In addition, the description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

Additional aspects of this disclosure are set out in the independent claims and preferred features are set out in the dependent claims. Features of one aspect may be applied to each aspect alone or in combination with other aspects. In addition, while certain operations in the claims are provided in a particular order, it is appreciated that such order is not required unless the context otherwise indicates.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system for medical image analysis, comprising:
a processor in communication with a memory, the memory including instructions, which, when executed, cause the processor to:
access data associated with one or more medical input images;
extract, using one or more models, features from the data associated with the one or more medical images, the one or more models trained to extract the features according to a self-supervised learning framework including discriminative learning, restorative learning, and adversarial learning; and
generate, using the features extracted from the one or more medical input images, visualizations of an area of interest suitable for disease localization.

2. The system of claim 1, wherein the model is trained by one or more processors, that:
access input patches x₁ and x₂ of a same class/instance;
apply random data augmentations T(·) to the input patches; and
provide the input patches as input to discriminative and restorative branches of the self-supervised learning framework, wherein the discriminative branch consists of twin encoders f_{θ} and f_{ξ}, and projectors hθ and hξ, and encodes the input patches to high-level embeddings vectors and trains the one or more models to maximize the agreement between the (high-level) embedding vectors of the samples from the same (pseudo) class.

3. The system of claim 2, wherein the restorative branch consists of an encoder f_{θ} and decoder gθ, and restore the original input patches from their distorted ones and trains the one or more models to maximize the (pixel-level) agreement between original samples and their restored versions.

4. The system of claim 2, wherein the self-supervised learning framework includes an adversarial branch that consists of a discriminator network Do and receives both the predictions of restorative branch original input samples as the input, the adversarial branch configured to engage in a minimax game with the restorative branch.

5. The system of claim 2, wherein the adversarial branch trains to distinguish between the original and restored samples, while the restorative branch attempts to confuse the adversarial discriminator by generating samples that are indistinguishable from real ones.

6. The system of claim 1, wherein by the discriminative learning the model is trained to model similarities among instances of a same (pseudo) class in an embedding space.

7. The system of claim 1, wherein the processor randomly reformats the one or more input medical images prior to feature extraction of the features.

8. The system of claim 1, wherein the model is trained using unlabeled medical image training data.

9. The system of claim 1, wherein the features are discriminative features captured throughout the one or more medical images that provide generalizable image representations.

10. The system of claim 1, wherein the model learns fine-grained representations, facilitating more accurate lesion localization with only image-level annotations.

11. The system of claim 1, wherein the features are highly reusable low/mid-level features, resulting in greater transferability to different medical tasks.

12. A method for medical image analysis, comprising:
accessing data associated with one or more medical input images;
extracting, using one or more models, features from the data associated with the one or more medical images, the one or more models trained to extract the features according to a self-supervised learning framework including discriminative learning, restorative learning, and adversarial learning; and
generating, using the features extracted from the one or more medical input images, visualizations of an area of interest suitable for disease localization.

13. The method of claim 12, further comprising:
training the one or more models according to the self-supervised learning framework, by:
accessing input patches x₁ and x₂ of a same class/instance;
apply random data augmentations T(·) to the input patches;
provide the input patches as input to discriminative and restorative branches of the self-supervised learning framework, wherein the discriminative branch consists of twin encoders f_{θ} and f_{ξ}, and projectors hθ and hξ, and encodes the input patches to high-level embeddings vectors and trains the one or more models to maximize the agreement between the (high-level) embedding vectors of the samples from the same (pseudo) class.

14. The method of claim 13, wherein the restorative branch consists of an encoder f_{θ} and decoder gθ, and restore the original input patches from their distorted ones and trains the one or more models to maximize the (pixel-level) agreement between original samples and their restored versions.

15. The method of claim 13, wherein the self-supervised learning framework includes an adversarial branch that consists of a discriminator network Do and receives both the predictions of restorative branch original input samples as the input, the adversarial branch configured to engage in a minimax game with the restorative branch.

16. The method of claim 13, wherein the adversarial branch trains to distinguish between the original and restored samples, while the restorative branch attempts to confuse the adversarial discriminator by generating samples that are indistinguishable from real ones.

17. A non-transitory, computer-readable medium storing instructions encoded thereon, the instructions, when executed by one or more processors, cause the one or more processors to perform operations to:
access data associated with one or more medical input images;
extract, using one or more models, features from the data associated with the one or more medical images, the one or more models trained to extract the features according to a self-supervised learning framework including discriminative learning, restorative learning, and adversarial learning; and
generate, using the features extracted from the one or more medical input images, visualizations of an area of interest suitable for disease localization.

18. The non-transitory, computer-readable medium of claim 17, wherein the adversarial learning is configured to improve feature learning through the restoration learning.

19. The non-transitory, computer-readable medium of claim 17, wherein the discriminative learning is configured to learn high-level discriminative representations.

20. The non-transitory, computer-readable medium of claim 17, wherein the restoration learning is configured to enforce the one or more models to conserve fine-grained information about a given input image by focusing on more localized visual patterns.
